(19) United States
(12) Patent Application Publication
(10) Pub. No.: US 2015/0185207 A1
(43) Pub. Date: Jul. 2, 2015

(54) Quantifying Neutrophil Concentration in Blood

(71) Applicants: Gordon Black, Glasgow (GB); Brian Wilson, Toronto (CA)

(72) Inventors: Gordon Black, Glasgow (GB); Brian Wilson, Toronto (CA)

(21) Appl. No.: 14/444,410

(22) Filed: Jul. 28, 2014

Related U.S. Application Data
(60) Provisional application No. 61/859,859, filed on Jul. 30, 2013.

(51) Int. Cl. G01N 33/50

(52) U.S. Cl. CPC G01N 33/5091

(57) ABSTRACT

The present invention comprises medical diagnostic methods and devices that quantify neutrophil populations in blood by using optical spectroscopy, either ex vivo with collected blood or non-invasively in vivo. In certain embodiments, fluorescent and Raman spectroscopy may be used to distinguish and/or quantify the neutrophils from the other blood components. The methods and devices of the invention advance the detection of sepsis by developing a point of care diagnostic device capable of rapid and/or real-time quantification of neutrophils. Other embodiments of the technology are also envisaged, particularly for analysing blood constituents both endogenous and administered.

### CROSS REFERENCE TO RELATED APPLICATIONS

This application claims priority to application 61/859,859 filed on Jul. 30, 2013. The 61/859,859 application is hereby incorporated by reference herein in its entirety.

### FIELD OF THE INVENTION

The present invention relates generally to methods and medical devices and more specifically it relates to the quantification of neutrophils in blood using optical spectroscopy.

### BACKGROUND OF THE INVENTION

This invention relates in general to systems and methods for quantifying blood components through optical spectroscopy. In one embodiment the blood components are neutrophils. The methods and devices for quantifying neutrophils according to the invention may be used in the diagnosis and monitoring of neutrophil related diseases such as sepsis and neutropenia.

### Sepsis

Sepsis is life threatening, systemic inflammation resulting from infection. Sepsis remains one of largest causes of mortality and morbidity in the world: for example, in the USA deaths from severe sepsis (>200,000 per year) exceed those of acute myocardial infarction and common cancers (Vincent et al., 2002). Early identification of sepsis is crucial for survival, as early treatment of sepsis/SIRS (systemic inflammatory response syndrome) is strongly correlated with positive clinical outcomes. Identifying physiological changes occurring in response to infection is crucial in decreasing morbidity and mortality. The earlier that sepsis is identified, the earlier treatment can be started. This has been proven to significantly improve patient outcomes. The importance of early treatment is acknowledged in the universal guidelines for sepsis treatment known as the Sepsis 6, i.e. six clinical actions that should be undertaken within a 1-hour target to improve rates of survival (Dellinger R P et al., 2013).

For every 1-hour delay of administration of antibiotics, mortality increases by 8% (Kumar et al., 2006; Levinson et al., 2011; www.survivingsepsis.org—accessed Jul. 16, 2014). Pneumonia, urinary tract infections and wound infections are a few examples of potentially fatal infections that can cause sepsis. Certain embodiments of the present invention enable the identification of sepsis in its early stages allowing treatment to be started early.

Four measurements are used to identify sepsis/systemic inflammatory response syndrome (SIRS). They are (1) Respiratory Rate, (2) Heart Rate, (3) Temperature and (4) White Cell Count. A patient is deemed to be septic if two or more of the four criteria are identified to be abnormal with evidence of infection (Moore et al, 2009). Early diagnosis of sepsis can be a challenge for a variety of reasons and individual patients can present very differently. Some of the challenges that contribute to delays in the diagnosis of sepsis include the body's ability to compensate and mask some of these signs. This is especially true in the young and in athletes. Also, medications can alter the body's response to infection. For example, beta-blockers decrease heart rate thereby masking the heart's response to infection. Furthermore, elderly patients as well as patients with medical co-morbidities can have difficulty mounting a physiological response to infection. These are just some of the difficulties faced that can obscure and delay the diagnosis and management of sepsis.

### Neutrophils

The normal range for the concentration of white cells in the blood in humans is 4.0-12.0×10⁹/L. Neutrophils account for the majority of the circulating white blood cells, having a normal range of 1.8-7.7×10⁹/L. Infection is suspected if the white cells are above 12.0×10⁹/L or neutrophils above 7.7×10⁹/L. Similarly, infection is possible if white cells are below 4.0×10⁹/L or neutrophils below 1.8×10⁹/L. Furthermore, a neutropenic patient is categorized as being severely immunocompromised if their neutrophils fall below 0.5×10⁹/L. A patient with less than this number of neutrophils is prone to infection. Neutropenic sepsis occurs when the neutrophil count falls below 0.5×10⁹/L and the patient has an infection. These conditions can be life threatening and require prompt recognition and initiation of treatment.

Neutrophils are produced in the bone marrow by haematopoiesis and are the most numerous white blood cells in the circulation. They account for 50-70% of circulating white cells in the absence of infection. Neutrophils play an important role in innate immunity and are the first white blood cells to respond and increase their circulating numbers to fight infection. This rise in neutrophils is known as a neutrophilia and is usually an indication of infection. It is the neutrophilia that is responsible for increased white cell count observed in infection.

Neutrophils are phagocytes, which is why their numbers increase in infection. The internalised phagosome can fuse with the bactericidal granules which fill the neutrophil cytoplasm. In a process called respiratory burst, oxygen is consumed by the neutrophils to produce reactive oxygen species (ROS) that are highly bactericidal. These cells contain large amounts of NADPH oxidase which are latent at rest. However, during infection NAPDH oxidase becomes activated, which reduces NADPH to form superoxide, a highly effective bactericidal ROS.

Neutrophils are a key part of the innate immune system and are important cells in responding to acute infection and inflammation. The value of monitoring their numbers in infection is acknowledged and is common practice. However, currently no device exists that can rapidly quantify circulating neutrophils.

Neutrophils are the most common white blood cells in peripheral blood and are crucial for innate immunity, since they are the first blood cells to increase in number in response to infection. Each neutrophil is packed with granules which help fight infection. These granules strongly autofluoresce when excited at specific wavelengths of light (Monici et al., 1995). The fluorescence arises from known fluorescent biomolecules found naturally in the cells. In addition, neutrophils have unique Raman (inelastic scattering) spectra that can be used to identify them from other blood components (Ramoji et al., 2013). Certain embodiments of the present invention utilize these optical properties of neutrophils to rapidly and accurately quantify neutrophils in the body's septic response.

Three of the four sepsis measurements are frequently monitored in patients as routine observations and recorded on early-warning charts. These charts are updated frequently and monitor the patient for signs of deterioration or improvement. The four sepsis measurements are also used to make a rapid assessment of patient's at presentation. White cell quantification is the only sepsis/SIRS measurement not routinely monitored. The problem is that white cell quantification is vital in diagnosing sepsis but the blood samples take hours to process instead of minutes or seconds. For example, hospital haematology laboratories require 1 hour to process urgent full blood counts and 4 hours to process non-urgent samples (Gill et al., 2012). In addition to this, white cell count is the only SIRS measurement that is an uncomfortable, invasive procedure as it involves either venapuncture or other blood letting.

### Fluorescence and Raman Spectroscopy

Fluorescence involves light absorption by molecules to generate excited electronic states, followed by re-emission of light at longer wavelengths. Many biomolecules in body fluids, cells and tissues fluoresce naturally (so-called endogenous or autofluorescence). The excitation spectra and the emission spectra depend on the molecular composition and, to a lesser extent on the physical and chemical environment. In addition to autofluorescence, a wide variety of fluorescent materials may be used to ‘label’ cells or tissues.

Inelastic or Raman scattering involves the exchange of energy between light photons and the vibrational or rotational states of molecules, in which a small amount of energy is gained or lost by the photons resulting in their having, respectively, shorter or longer wavelength than the incident photons (Schie and Huser, 2013). Hence, if the sample of interest is illuminated with monochromatic light, the small wavelength shifts can be detected to generate a corresponding Raman spectrum. In biomolecules the Raman signal is generally much weaker than the autofluorescence, depending on the molecules and the wavelengths used. However, the fluorescence emission spectrum of most biomolecules is relatively broad; typically tens of nanometers and shows limited structure. By contrast, Raman spectra of biomolecules are typically complex with multiple peaks (lines) that are only few nanometers wide. Hence, the Raman spectra or different biomolecules are usually highly specific, enabling so-called “fingerprinting” to distinguish between molecules. Raman spectra are often presented in terms of the frequency or wavenumber shift rather than wavelength, but these quantities are directly related. Near-infrared absorption spectroscopy is often used as an alternative to Raman spectroscopy but the high light attenuation by water limits some biomedical applications, particularly in vivo and hydrated samples.

### Fluorescence and Raman Spectra of Neutrophils.

In intact cells such as neutrophils the contributions from several or many molecules comprise the overall combined fluorescence or Raman characteristics. Several studies have shown that neutrophils are autofluorescent. For example, Monici et al. (1995), measured the fluorescence emission spectra of white blood cells ex vivo. Heintzelman et al. (2000) also measured the autofluorescence of polymorphonuclear and mononuclear leukocytes and cervical endothelial cells. Peak excitation wavelengths were are 290, 350, 450 and 500 nm, while the corresponding emission spectra showed single peaks at around 330, 450, 530 and 530 nm, respectively. They attributed these spectral characteristics as due to tryptophan, NAD(P)H, FAD and an unknown fluorophore, respectively. These findings were performed and presented only in the context of discriminating inflammation from dyspasia for cancer diagnostics.

The fluorescence excitation and emission spectra of neutrophils are most intense at relatively short wavelengths, in the UV or blue region of the spectrum. Raman scattering occurs across a wide wavelength range, including into the near-infrared above about 700 nm. Hemoglobin in blood has a complex optical absorption spectrum that is highest in the long-UV and short-visible range (between about 350 and 450 nm) and decreases above about 600 nm (http://omlc.ogi.edu/spectra/hemoglobin—accessed Jul. 16, 2014)). This absorption reduces the intensity of the fluorescence or Raman light that can be detected from the other cells such as neutrophils. As a result, the choice between using the neutrophil fluorescence or Raman scattering, and the corresponding optimum excitation and detection wavelengths is a trade-off between several factors, including: the strength of the intrinsic optical signals; the excitation and detection wavelengths to give optimal optical signals from the cells; the degree of attenuation of the delivered and detected light in the blood sample or in tissue containing the cells; and the available light sources, spectral analyzers and photodetectors, and the performance characteristics of these components.

In general the optical absorption due to haemoglobin has greater impact on measuring the autofluorescence of neutrophils than on measuring their Raman scattering, since the Raman measurements can be made in the near-infrared wavelength range where the optical absorption of blood is reduced compared to shorter UV or visible wavelengths of autofluorescence. Hence, for ex vivo measurements in blood, the present invention includes a means to remove the hemoglobin from the sample before a fluorescence measurement is made. This may be used also for Raman measurements to improve the signal-to-background ratio.

Fluorescent spectroscopy is a technique utilized to identify or accurately quantify a substance. However, many substances do not fluorescence or are weakly fluorescent or florescence only at wavelengths that are not suitable for the intended purpose. Hence, it is common to incubate the targeted substance, such as cells, with a laboratory-manufactured fluorescent marker. These markers include fluorescent dyes, activatable molecular beacons and fluorescent nanoparticles. Cells may also be modified to express fluoresecnt proteins. The markers may be targeted to cells of interest by attaching them to antibodies, peptides, aptamers or other moieties that are specifically taken up by or bind preferentially to the cells. This method also usually requires excess unbound reporters to be washed away. These approaches are used widely in biomedical research and for clinical diagnostics. Existing techniques include fluorescent-activated cell sorting (FACS) and fluorescent in-situ hybridisation (FISH). The fluorescence may also be used to image cells or tissues, either in ex vivo samples or in vivo in animal models.

In vivo fluorescence spectroscopy and imaging are also used clinically, either for disease detection or to guide interventions. For example, Valdes et al. (2012) and several other groups have reported the use of fluorescence spectroscopy and imaging for guiding resection of tumors such as gliomas using fluorescent markers or compounds that lead to the synthesis of fluorescent markers in the body. The disadvantages of quantifying cells or tissues with fluorescent markers are (i) the biomarkers must be manufactured, which can be laborious and costly, (ii) the biomarkers must incubate with the sample to bind to the target, which can be time consuming, and (iii) in vivo applications may be confounded by the need to delivery the marker to the cells or tissue of interest and there may also be potential toxicities.

Fewer applications have used autofluorescence to detect or measure target substances. Nevertheless, autofluorescence detection has been used to identify cancerous/pathological tissue from healthy tissue. For example, autofluorescence endoscopy is an established and commercial method used in the lung and gastrointestinal tract, as for example in the work of Goetz's (2013). Mehrotra et al., (2011) used autofluorescence to distinguish oral cancers from healthy tissue.

Neutrophil autofluorescence has been utilized to identify pathology. Heinztelman et al., (2000) used neutrophil fluorescence to identify cervical dysplasia to allow prompt management to guide cancer treatment. Monsel et al. (2014), used neutrophil autofluorescence from brochioalveolar lavage to diagnose pneumonia using microscopy. This study illustrated that neutrophil autofluorescence can be used to accurately diagnose infection, although this paper differs significantly from the present invention, since Monsel et al. measure activated neutrophils from the site of infection (brochioalveolar lavage) and identified the neutrophils ex vivo using microscopy. Although both papers showed a role for neutrophil autofluorescence in diagnosis, neither paper measured peripheral blood neutrophils nor indicated this approach.

Dorward et al. (2013) used neutrophil autofluorescence with FACS to separate neutrophils in blood samples. Neutrophils were completely separated from the other cells and molecules in the blood during FACS and then counted a single cell at a time. Significant processing was required to isolate the neutrophils from other blood components. The authors state that the time between initial withdrawing of the blood sample from the patient and obtaining purified neutrophils by FACS is approximately 3 hours. They did not indicate means to measure the neutrophil autofluorescence using lysis to remove the hemoglobin and then measuring the autofluorescence on the remaining cell sample. They did not indicate the use of autofluorescence measurements in vivo.

Zeng et al. (2014) used 2 photon autofluorescence flow-cytometry to measure in-vivo neutrophils in zebra fish. In this approach the fluorescence is excited using high-intensity pulsed light. These measurements were done in single vessels at a time in a non-mammalian species for the purpose of assessing the biological response to local thermal injury. Zebra fish are used in biomedical research because they are relatively transparent, so that the results of these studies do not translate into the ex vivo or in vivo methods used in the present invention. While Zheng et al used neutrophil autofluorescence in an animal in vivo setting, they did not indicate the concept of using this for the purpose of non-invasive assessment of sepsis in patients. Zeng et al., (2013) used 2 photon excitation of in vivo human leukocytes for functional imaging. They used this as an imaging technique and not for quatification.

Raman spectroscopy is commonly used to characterize biomaterials in many different fields, from quality control to chemical analysis to assessing art works and artefacts. A number of studies have reported the use of Raman spectroscopy in vivo, including in patients as a means to detected disease such as early cancer, for example the work of Kallaway et al. (2013) or Shim et al. (1997). Typically, these methods measure the whole Raman spectrum from the tissue and then use chemometric or similar techniques to “train” algorithms against gold-standard diagnosis from histopathology and these algorithms are then used subsequently in other patients to make a diagnosis. Raman microscopy is also available to map the Raman signatures of cells and tissues (Schie and Huser, 2013). Generally, Raman imaging is generally very slow compared to fluorescence imaging because of the relatively weak signals. Raman spectroscopy has been reported for non-invasive measurements of blood glucose for the purpose of monitoring diabetes (Dingari et al., 2011).

Tiba et al. (2014) used resonance Raman spectroscopy and near-infrared spectroscopy to monitor tissue haemoglobin saturation during haemorrhage in pigs. Measurements were made from the buccal mucosa and the forelimb. The authors showed the potential for accurate in-vivo measurements using Raman spectroscopy. However, they did not discuss using this method to identify the neutrophils or white blood cells in the blood, either in vivo or ex vivo. Neither did they indicate the use of pulsation or applied pressure to isolate the Raman signal coming from circulating blood against the signals coming from other tissue components.

Raman and infrared spectroscopy have been used to identify specific cell populations and/or molecules. Ramoji et al. (2012) showed that Raman spectroscopy could accurately differentiate leukocyte subtypes. In particular, they showed that neutrophils have a unique Raman fingerprint that distinguishes them from other white blood cell populations. This paper does not discuss the use of Raman spectroscopy for the purpose of measuring neutrophil concentration in blood, either ex vivo or in vivo.

Pulse oximetry uses non-invasive optical measurement of pulsatile blood to measure oxygen saturation of haemoglobin. Pulse oximeters are common devices used in the clinical setting that provide important information on the percentage of haemoglobin bound to oxygen, which is used to assess the patient status. For example, pulse oximetry can help identify patients in respiratory failure and help monitor anaesthetised patients. A drop in oxygen saturation could be due to a variety of conditions including pneumonia, pulmonary embolism, pulmonary oedema, asthma, COPD, pneumothorax or pleural effusion. Pulse oximeters work by measuring the light that is diffusely reflected from or transmitted through tissue, such as a finger, toe or earlobe. This diffuse light has undergone multiple elastic scattering interactions in the tissue. These interactions are to be distinguished from the inelastic Raman scattering. Some of the light may also be absorbed, including by haemoglobin in blood. Hence, the spectrum of the detected diffuse light shows the characteristic haemoglobin features, with dips in the spectra corresponding to peaks in the haemoglobin absorption spectrum. Since the absorption spectra of deoxyhemoglobin (Hb) is different from that of oxyhemoglobin (HbO₂), the oxygen saturation, SO₂═[HbO₂]/{[Hb]+[HbO₂]} can be estimated by deriving the concentrations of Hb and HbO₂ from the measured diffuse spectra: the square brackets here, [ ], indicate concentrations.

Automated cell counters use a lytic reagent to specifically lyse red blood cells after initial counting with electrical impedance, leaving white cells and platelets. The white cells and platelets are then analysed by putting the solution through a second count using electrical impedance. The white cells and platelets are distinguished by their size. Passing a dilute suspension of cells though a capillary tube one at a time and optically analysing single cells can obtain a white cell differential. This is a lengthy process, with urgent samples taking over 1 hour to process in the laboratory (Gill et al., 2012). Due to technical challenges, automated cell counting analysis is performed in the laboratory by a haematology technician, and generally not as a point of care test.

U.S. Pat. No. 4,883,055 describes an artificially induced blood pulse for use with pulse oximetry. No mention is made of application of a pressure probe(s) to modulate blood flow. Furthermore, no mention is made of use with neutrophil quantification. Additionally, no mention is made of diagnosing infection/sepsis.

U.S. Pat. Nos. 7,254,432 and 7,313,425 describe non-invasive optical measurements using transmission-mode and reflectance-mode and using 2 or more wavelengths of light for non-invasive measurements. The patents are primarily aimed at non-invasively quantifying haemoglobin. No mention is made on how an entire measurement is performed. Additionally, no mention is made of using Raman or fluorescence spectroscopy to quantify blood parameters. Furthermore, no mention is made of quantifying neutrophils. Also, no mention is made of diagnosing infection/sepsis.
Berger T, Green J, Horeczko T, Hagar Y, Garg N, Suarez A, Panacek E, Shapiro N. Shock index and early recognition of sepsis in the emergency department: pilot study. West J Emerg Med. 2013 March; 14:168-74.
Bogaards A, Sterenborg H J, Trachtenberg J, Wilson B C and Lilge L, In vivo quantification of fluorescent molecular markers in real-time by ratio imaging for diagnostic screening and image-guided surgery. Lasers Surg Med. 2007; 39:605-613
Dellinger R P, Levy M M, Rhodes A, Annane D, Gerlach H, Opal S M, Sevransky J E, Sprung C L, Douglas I S, Jaeschke R, Osborn T M, Nunnally M E, Townsend S R, Reinhart K, Kleinpell R M, Angus D C, Deutschman C S, Machado F R, Rubenfeld G D, Webb S A, Beale R J, Vincent J L, Moreno R; Surviving sepsis campaign: international guidelines for management of severe sepsis and septic shock: 2012. Crit Care Med. 2013; 41:580-637
Dickinson M E, Bearman G, Tille S, Lansford R, Fraser S E, Multi-spectral imaging and linear unmixing add a whole new dimension to laser scanning fluorescence microscopy. Biotechniques. 2001; 31):1272-1278
Dingari N C, Barman I, Singh G P, Kang J W, Dasari R R and Feld M S, Investigation of the specificity of Raman spectroscopy in non-invasive blood glucose measurements. Anal Bioanal Chem. 2011; 400:2871-2880
Doornbos R, Lang R, Aalders M, Cross F and Sterenborg H, The determination of in vivo human tissue optical properties and absolute chromophore concentrations using spatially resolved steady-state diffuse reflectance spectroscopy, Phys. Med. Biol. 1999; 44: 967-981
Dorward D A, Lucas C D, Alessandri A L, Marwick J A, Rossi F, Dransfield I, Haslett C, Dhaliwal K, Rossi A G. Technical advance: autofluorescence-based sorting: rapid and nonperturbing isolation of ultrapure neutrophils to determine cytokine production. J Leukoc Biol. 2013; 94:193-202.
Gill D, Galvin S, Ponsford M, Bruce D, Reicher J, Preston L, Bernard S, Lafferty J, Robertson A, Rose-Morris A, Stoneham S, Rieu R, Pooley S, Weetch A, McCann L. Laboratory sample turnaround times: do they cause delays in the E D? J Eval Clin Pract. 2012; 18:121-7
Goetz M. Real-time histology in colonoscopy. Gastroenterol Clin North Am. 2013 September; 42:567-75.
Heintzelman D L, Lotan R, Richards-Kortum R R. Characterization of the autofluorescence of polymorphonuclear leukocytes, mononuclear leukocytes and cervical epithelial cancer cells for improved spectroscopic discrimination of inflammation from dysplasia. Photochem Photobiol. 2000; 71:327-32.
Kallaway C, Almond L M, Barr H, Wood J, Hutchings J, Kendall C, Stone N. Advances in the clinical application of Raman spectroscopy for cancer diagnostics. Photodiagnosis Photodyn Ther. 2013; 10:207-19.
Kim A, Khurana M, Moriyama Y and Wilson B C, Quantification of Fluorescence Decoupled From Optical Properties Effects Using Fiberoptic Reflectance Measurements, J Biomed Optics 2010; 15: 067006
Kumar A, Roberts D, Wood K E. Duration of hypotension prior to initiation of effective antimicrobial therapy is the critical determinant of survival in human septic shock. Crit Care Med 2006; 34:1589-96.
Levinson A T, Casserly B P, Levy M M. Reducing mortality in severe sepsis and septic shock. Semin Respir Crit Care Med. 2011; 32:195-205.
Li Y, Zhou X and Ye D, Molecular beacons: an optimal multifunctional biological probe, Biochem Biophys Res Commun. 2008; 373:457-461.
Mehrotra R, Gupta D K. Exciting new advances in oral cancer diagnosis: avenues to early detection. Head Neck Oncol. 2011; 3:33.
Monsel A, Lécart S, Roquilly A, Broquet A, Jacqueline C, Mirault T, Troude T, Fontaine-Aupart M P, Asehnoune K. Analysis of autofluorescence in polymorphonuclear neutrophils: a new tool for early infection diagnosis. PLoS One. 2014; 9: e92564.
Monici M, Pratesi R, Bernabei P A, Caporale R, Ferrini P R, Croce A C, Balzarini P, Bottiroli G. Natural fluorescence of white blood cells: spectroscopic and imaging study. J Photochem Photobiol B. 1995; 30:29-37.
Moore L J, Jones S L, Kreiner L A, McKinley B, Sucher J F, Todd S R, Turner K L, Valdivia A, Moore F A. Validation of a screening tool for the early identification of sepsis. J Trauma. 2009; 66:1539-46
Müller M G, Georgakoudi I, Zhang Q, Wu J, Feld M S, Intrinsic fluorescence spectroscopy in turbid media: disentangling effects of scattering and absorption. Appl Opt. 2001; 40:4633-4646
http://omlc.ogi.edu/spectra/hemoglobin
Ramoji A, Neugebauer U, Bocklitz T, Foerster M, Kiehntopf M, Bauer M, Popp J. Toward a spectroscopic hemogram: Raman spectroscopic differentiation of the two most abundant leukocytes from peripheral blood. Anal Chem. 2012; 84:5335-42.
Schie I W and Huser T, Label-free analysis of cellular biochemistry by Raman spectroscopy and microscopy, Compr Physiol. 2013; 3:941-956
Shim M G and Wilson B C, Development of an In Vivo Raman Spectroscopy System for Diagnostic Applications, J Raman Spect 1997; 28: 131-142
Schie I W, Huser T. Label-free analysis of cellular biochemistry by Raman spectroscopy and microscopy. Compr Physiol. 2013; 3:941-56.
Surviving Sepsis Campaign. www.survivingsepsis.org
Tiba M H, Draucker G T, Barbee R W, Terner J, Filho I T, Romfh P, Vakhshoori D, Ward K R. Tissue oxygenation monitoring using resonance Raman spectroscopy during hemorrhage. J Trauma Acute Care Surg. 2014; 76:402-408.
Valdes P A, Leblond F, Kim A, Wilson B C, Paulsen K D and Roberts D W, A Spectrally-Constrained Normalization Technique for Protoporphyrin IX Quantification in Fluorescence-Guided Surgery, Optics Lett 2012; 37:1817-1819
Vetromile C M and Jameson D M, Frequency domain fluorometry: theory and application, Methods Mol Biol. 2014; 1076:77-95
Vincent J L, Abraham E, Annane D, Bernard G, Rivers E, Van den Berghe G.
Reducing mortality in sepsis: new directions. Crit Care. 2002; 6 Suppl 3:S1-18.
Zeng Y, Yan B, Sun Q, He S, Jiang J, Wen Z, Qu J Y. In vivo micro-vascular imaging and flow cytometry in zebrafish using two-photon excited endogenous fluorescence. Biomed Opt Express. 2014; 5:653-63.
Zeng Y, Yan B, Sun Q, Teh S K, Zhang W, Wen Z, Qu J Y. Label-free in vivo imaging of human leukocytes using two-photon excited endogenous fluorescence. J Biomed Opt. 2013 April; 18(4):040504.

### BRIEF DESCRIPTION OF THE DRAWINGS

In order to understand the invention and to see how it may be carried out in practice, various embodiments will now be described, by way of non-limiting examples only, with reference to the accompanying drawings, in which:

FIG. 1 illustrates near-infrared Raman spectra taken of components of blood ex vivo, including neutrophils, red blood cells and plasma, in which the intensity of the Raman signal is plotted as a function of the chemical shift in the range 400 to 4000 cm⁻¹;

FIG. 2 graphically illustrates an embodiment of the invention showing the main principles of making optical measurements on a body part (the finger in this example) using a probe that also applies mechanical pressure to alter the blood content of the body part for the purpose of isolating the optical signal coming from components of the blood;

FIG. 3 shows an example of the diffuse reflectance spectrum measured in vivo on the finger of an adult human subject with and without the application of local mechanical pressure to the finger nail as would be performed as illustrated in FIG. 2;

FIG. 4 graphically illustrates one example of the main optical elements of one embodiment of a device used in combination with the device illustrated in FIG. 2 to measure the fluorescence or Raman spectra from a body part in vivo and also to measure the diffuse transmittance of the body part in vivo;

FIG. 5 graphically illustrates an embodiment of the device to quantify neutrophils in an ex vivo blood sample using a cassette that incorporates both a filter element to separate the neutrophils from the blood and remove the hemoglobin and plasma and optical windows through which spectroscopic measurements can be made of the neutrophils trapped on the filter;

FIG. 6 graphically illustrates one example of the main optical elements of one embodiment of a device used in combination with the device illustrated in FIG. 5 to measure the fluorescence or Raman spectra of neutrophils in a blood sample.

### DETAILED DESCRIPTION OF THE INVENTION

The present invention comprises medical diagnostic methods and devices that detect and quantify neutrophil populations in blood by using optical spectroscopy. Embodiments include ex vivo devices and methods with collected blood or non-invasively in vivo. In certain embodiments, fluorescent and Raman spectroscopy may be used to distinguish and/or quantify the neutrophils from the other blood components. The methods and devices of the invention advance the detection of sepsis by developing a point of care diagnostic device capable of rapid and/or real-time quantification of neutrophils. Other embodiments of the technology are also envisaged, particularly for analysing blood constituents both endogenous and administered.

In certain embodiments, the current invention seeks to overcome the limitations of existing sepsis monitoring by methods and device capable of rapid or real-time quantification of neutrophils using optical spectroscopy or spectral measurements. This will advance the detection of sepsis and promptly identify patients in need of urgent treatment, as well as enabling longitudinal monitoring or patients over time to assess response to treatment. The value of this diagnostic instrument is that it will complete the assessment of a patient's septic status, as all 4 sepsis criteria could now be monitored routinely (Respiratory Rate, Heart Rate, Temperature & White Cell Count). The methods and devices are based on in vivo or ex vivo quantification of neutrophils. They may also be used to assess other blood constituents.

In certain embodiments, the present invention utilises the changes in the diffuse reflectance or transmittance spectra of light in vivo due to changes in the blood content in tissue. To our knowledge no method or device has been reported that uses these measurements to isolate the fluorescence or Raman signals from blood components, including neutrophils, in order to quantify these components. The methods and devices for in vivo measurement of neutrophils in blood may use either the natural or induced changes in the blood content within the body part in order to isolate the fluorescence or Raman signals originating in the blood from the background signals coming from the other tissue components. The neutrophil concentration in the blood is then determined using their fluorescence or Raman spectral characteristics. Making these measurements quantitative may also utilize information on light attenuation of the fluorescence or Raman signals in the tissue obtained by measurements of the diffuse reflectance or transmittance of the tissue.

The methods and devices of the present invention will allow rapid or real-time monitoring of white cells and shorten time to treatment. One goal of certain embodiments of this invention is to identify sepsis at its early stages to save lives and to enable monitoring of the patient's response to treatment.

An additional advantage of certain embodiments of this invention will be to reduce the overuse of antibiotics by rationalizing therapeutic decisions, thereby reducing antibiotic resistance and costs.

In view of the limitations of current neutrophil quantification known in the art, certain embodiments of the present invention describe new diagnostic methods and devices that can be utilized for increasing the speed of quantification of neutrophils. In certain embodiments, this may be done non-invasively.

Certain embodiments of the methods and devices of the present invention include one of two approaches. In the first (ex vivo) approach, spectroscopic fluorescence or Raman measurements are made on of blood samples taken from the patient. In the second approach (in vivo), spectroscopic measurements are made on a body part in a non-invasive or minimally-invasive manner. In both approaches, additional enabling procedures are used in combination with the fluorescence or Raman spectroscopic measurement in order to improve the detectability of the neutrophil fluorescence or Raman signals and so achieve accurate measurement of the neutrophil concentration in blood. The spectroscopic identification of neutrophils based on their fluorescence or Raman spectral characteristics may use characteristics that are known in the literature or may use additional characteristics.

### Ex Vivo Approach:

For the ex vivo approach, the enabling procedures may, but do not necessarily, include the lysis of the red blood cells for the purpose of removing or substantially reducing the haemoglobin content by filtration or other means. The blood plasma may also be removed. The fluorescence or Raman spectra of the resulting cells, comprising the white blood cells and platelets, are then obtained using established spectroscopic techniques. In the case of removing the haemoglobin component by filtration, these spectroscopic measurements may be taken with the cells, including neutrophils, remaining trapped on the filter. In certain embodiments, the invention includes a device in which one or more size filters are integrated into a cassette that has one or more optical windows through which optical measurements can be made. The spectra are then used as input to a spectroscopic algorithm to identify the characteristic signature of neutrophils and separate this from other contributions to the spectra. These algorithms include but are not limited to principal component analysis, neural networks and spectral decomposition. The result is an estimate of the neutrophil concentration in the blood sample. This measurement may replace the current clinical-laboratory measurement that uses techniques such as electrical impedance.

Certain benefits of the invention are that the procedure can be substantially automated, so that minimal processing of the blood sample is required by the nurse or physician, and that the result is obtained rapidly. Hence, in certain embodiments, the devices according to the invention can be used at the patient's bedside or in other clinical settings.

### In Vivo Approach.

For in vivo approaches according to certain embodiments of the invention, non-invasive measurements may be made on a convenient body part, such as a finger. The fluorescence or Raman spectra are obtained by illuminating the body part with light of appropriate wavelengths and collecting the light that exits the body part and detecting the light after it has passed through appropriate spectral filters or a spectrometer. The enabling procedures include simultaneously measuring light that is diffusely reflected from or transmitted through the body part while the blood volume in the body part is varying in time. The time varying blood content may be due to the natural pulsatile flow of blood or due to applied pressure or other means. This information is then used, together with the time dependence of the fluorescence or Raman measurements to isolate the fluorescence or Raman signals originating in the blood from the signals coming from other tissue components. The purpose is to increase the signal-to-background ratio of the measurement. As in the case of the ex vivo approach, spectral analysis may be applied to separate the fluorescence or Raman signals originating in the neutrophils from those of other blood components including, in the in vivo embodiments, the red blood cells and blood plasma. A further enabling aspect of the invention is to use the diffuse light signals, or other modified diffuse light measurements, to correct the measured fluorescence or Raman signals coming from the blood for the effects of light absorption and elastic scattering through the body part. The correction may be applied at the fluorescence excitation and emission wavelengths or at the wavelengths of the incident and detected Raman light or at other wavelengths such as the Hb-HbO₂ isobestic point. The correction then allows the concentration of the neutrophils in the circulating blood to be calculated.

Features of certain embodiments of the in vivo methods and devices according to the invention are, firstly, that the measurements do not require a blood sample to be taken. However, in certain embodiments, it may be preferred to make an initial ex vivo measurement, either by standard clinical-laboratory assays or by the ex vivo approach of the present invention, to calibrate or normalize the in vivo measurement in each patient. Secondly, in certain embodiments the measurements may be made very rapidly, in real-time or near real-time, and may be made continuously or at frequent intervals, so that the status of the patient may be monitored, either as the condition progresses or in response to treatment. Thirdly, devices according to the invention may be configured to be suitable for bedside use or other clinical settings, even in patients who are unable to cooperate.

For embodiments comprising non-invasive in vivo measurements, the present invention may include a means to isolate either the autofluorescence or Raman signals from the blood from the signals from other tissues, based on varying the blood content of the body part being optically interrogated. The term “non invasive” here includes the idea that the optical measurements are made using external light sources and photodetectors. However, it would also be possible to use a “minimally invasive” approach in which, for example, small-diameter optical fibers are inserted into the body part. The present invention also includes various means to reduce the effect of haemoglobin absorption on the neutrophil measurement in vivo. As in the case of the ex vivo approach, it is expected that this will have greater impact on autofluorescence measurements than on Raman measurements, especially where the latter are made in the near-infrared spectral region.

A further aspect of certain embodiments of the invention is to make the fluorescence or Raman measurement quantitative, so that the concentration of the neutrophils in the blood can be estimated. For this purpose the in vivo measured fluorescence or Raman signals are corrected for the effects of light attenuation (absorption and elastic scattering) by the tissue. This is a well-known problem in the field of biomedical optics and the present invention may take advantage of a variety of methods and devices that have been described to solve these issues (Muller et al., 2001; Bogaards et al., 2007). These methods and devices may be grouped into two main classes.

In the first class the primary purpose is to determine the intrinsic autofluorescence or Raman spectrum of the tissue, usually for disease diagnosis such as cancer or for therapeutic guidance. For example, the main absorption peaks of hemoglobin cause dips in the measured fluorescence spectrum from tissue and this can cause artefactual peaks in the fluorescence spectrum that may be mistaken for real peaks. In this case, the clinical or biological information lies in detecting the so-called “intrinsic spectrum” of fluorescence from the tissue, undistorted by these absorbing and scattering effects. Differences in the intrinsic spectra between diseased and normal tissues are then used for diagnosis, This approach has been described in the work of Muller et al. (2001), for example. In general, distortion of the spectrum by absorption and elastic scattering is less of a problem for in vivo Raman spectroscopy, since the Raman spectrum is typically spread across a much narrower range of wavelengths (typically tens of nm) than the fluorescence spectra (typically, hundreds of nm). The use of different algorithms allows these methods to be employed in the present invention.

In the second class of “correction” methods and devices that may be used in certain embodiments of the present invention a primary purpose of the reported methods for fluorescence quantification in vivo is to estimate the tissue concentration of one or more exogenous fluorophores, such as an administered dye or photosensitizer. This may be done in one of two distinct methods and devices. In the first method, a semi-empirical ratiometric technique is used, such as those reported by Bogaards et al. (2007). In this technique two or more fluorescence excitation or emission wavelengths are employed and ratios of the intensity of the detected fluorescence signals at these wavelengths are taken and applied to the measured fluorescence signal. This corrects, in part, for factors such as the light absorption and scattering in the tissue, the light source and light detector distances and orientations with respect to the tissue and to each other, and the background tissue autofluorescence. In some cases the diffuse reflectance spectrum or the diffuse reflectance at one or more wavelengths is also included in the ratiometric calculation.

In an alternative approach, reported most notably by Kim and colleagues (2010) is a fiberoptic probe device for measuring a fluorescent photosensitizer concentration in tumor tissue during surgery. These authors have reported its successful use in guiding brain tumor resection. The principle of the method is to measure the fluorescence spectrum from the tissue (which includes the unknown concentration of the photosensitizer or other administered dye) and also the diffuse reflectance spectrum. The latter spectra are measured at two different separations of the optical fiber delivering broad-band light from the light source to the tissue and the optical fiber that collects the scattered light and transferred it to a spectral detector. An algorithm is then used in which the diffuse reflectance spectrum at each source-detector separation is fitted to a diffusion theory model of light propagation in tissue, using as inputs the known optical absorption coefficient spectra of haemoglobin (Hb and HbO₂), together with a simple mathematical form for the wavelength dependence of the transport scattering coefficient of the tissue. This so-called “spectrally constrained” technique enabled the absorption and transport scattering coefficient spectra of the tissue to be calculated using the information at the two source-detector separations. These coefficients were then applied, also using diffusion theory, to correct the measured fluorescence for the attenuation of the fluorescence excitation and emitted light in order to estimate the summed intrinsic plus photosensitizer fluorescence spectrum. Subtracting the autofluorescence allowed the concentration of the photosensitizer to be estimated, to a cited accuracy of about +/−10%, knowing its extinction spectrum (absorption coefficient per unit concentration).

Kim's approach may be modified to be used in the present invention if the following differences are accounted for. Firstly, at the short excitation and emission wavelengths of the neutrophil fluorescence, diffusion theory may not be sufficiently accurate in tissue to correct in this way for the effects of tissue attenuation in order to calculate the neutrophil concentration. Secondly, the present invention is not based on measuring the full fluorescence and diffuse reflectance spectra in order to implement the spectrally-constrained model used by Kim and colleagues. Thirdly, the present invention does not necessarily use two different source-detector separations. Fourthly, the present invention does not necessarily describe a method or device to estimate the concentration of an exogenous fluorophore, except in the case where neutrophil-specific dyes, molecular beacons or nanoparticles are used rather than the endogenous fluorescence of these cells. Lastly, the present invention is generally concerned with the time-dependent changes in the blood content of the tissue discussed above and the effect of this on the measured diffuse reflectance from or transmittance through the tissue as additional information that is applied to correct for the light attenuation by the blood.

This last point generally distinguishes the present invention from both classes of fluorescence quantification technique (ratiometric and spectrally-constrained) and is equally applicable to Raman-based measurements. Nevertheless, one or more of ratiometric or spectrally-constrained methods could be applied to certain embodiments of the present invention to deal with the problem of quantifying the neutrophil content from the fluorescence or Raman signals measured in the blood, following the isolation of these signals in the blood from background signals from other tissue components as described in the present invention.

### In Vivo Neutrophil Quantification

Certain embodiments of the invention include the use of the Raman spectroscopy to measures neutrophils in blood non-invasively. Raman scattering from biomolecules may occur across a wide spectral range, from the UV to the visible to the infrared, the wavelength (λ) dependence varying approximately as 1/λ⁴.

In one embodiment, a light source of one or more wavelengths suitable to generate Raman light from the neutrophils is used to illuminate a body part. The use of near-infrared light for this purpose has specific advantages: it has deeper penetration though tissue, allowing the blood to be sampled over a larger volume than at shorter wavelengths, it is less affected by light attenuation by hemoglobin and other tissue components, and the background autofluorescence from tissues is reduced. However, shorter wavelengths may also be used if it is desired to confine the effective tissue sampling volume to shallower depths, as for example in the use of local pressure.

Some fraction of the inelastically-scattered photons exit from the body part and may be collected by suitable optical elements. Light that is elastically scattered without being absorbed in the tissue is also present and is first removed by a suitable filter, such as a notch filter or cut-on filter. (A cut-on filter is used when the Stokes Raman light is to be measured: the anti-Stokes light may also be used, in which case a filter is selected to remove the elastically-scattered light, while passing or reflecting the shorter-wavelength inelastically-scattered light.) The Raman light may then be passed through one or more discrete optical filters to identify selected Raman “lines” or may be passed through a spectrometer to be spectrally resolved across a spectral range that encompasses all or selected portions of the full Raman spectrum. The Raman spectra of biomolecules and cells typically have very narrow and discrete peaks (“lines”) on the order of a few nanometers width and located at specific wavelengths. Since the lines are shifted by a constant energy from the incident light, it is common in Raman spectroscopy to express the spectra in wavenumbers in units of cm⁻¹ rather than directly in wavelengths as used in fluorescence spectroscopy.

The foregoing general methods for performing in vivo Raman spectroscopy are well known and can be implemented using open-beam optics or optical fibers, or a combination thereof.

The Raman spectra or the Raman signals at selected wavenumbers may be used to identify the specific signatures associated with neutrophils from other components in the blood or tissue. The present invention includes means by which the Raman signals originating from the circulating blood are isolated from those from other non-time-varying tissue components. In certain embodiments, this allows the problem to be significantly simplified, by reducing the analysis to that of separating the neutrophil Raman signals only from the signals of other blood components.

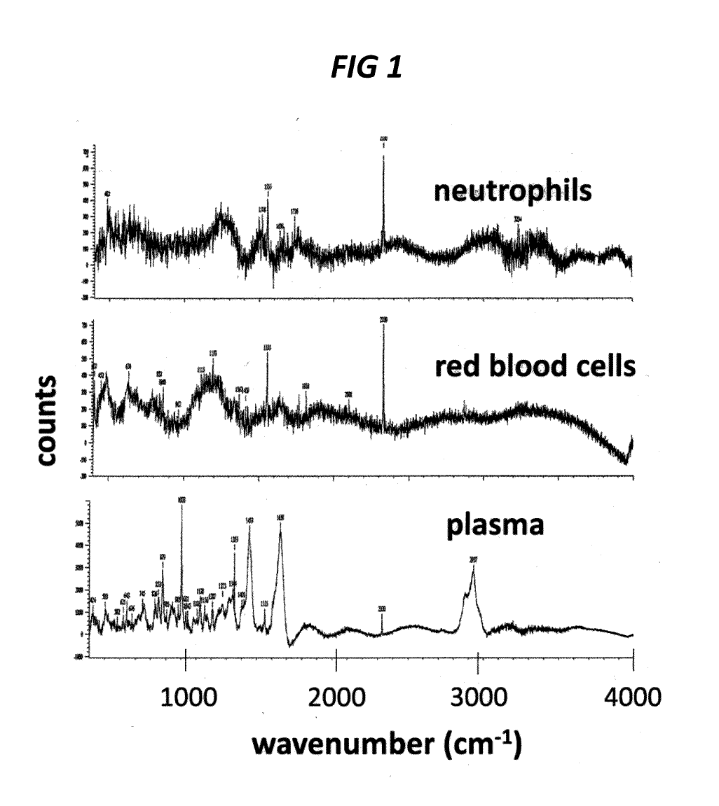

The ability to differentiate between neutrophils and other blood components is demonstrated in FIG. 1, which shows the Raman spectra of mammalian neutrophils, red blood cells and blood plasma measured ex vivo. As seen in FIG. 1, the three spectra are distinctly different. Hence, in the in vivo embodiment of the invention spectral analysis can be applied to differentiate the signal from white blood cells, including neutrophils, from those of the red blood cells and the plasma. This is the critical capability according to the invention in order to monitor neutrophils in the ciculating blood in vivo for the purpose of diagnosing sepsis and other conditions where the neutrophil concentration is abnormal.

Since the Raman spectra of the neutrophils and other blood components are distinct, there are several approaches according to the invention for separating the Raman signals of neutrophils from those of the other blood components. These utilize other embodiments of the invention whereby the Raman or fluorescence signals from blood in vivo are also isolated from the signals from other tissue components. One approach according to the invention is to assume that the spectral features that identify neutrophils and those that identify other blood cells and blood plasma are sufficiently constant between patients that population-averaged spectra of each component can be used as reference standards or basis spectra. Known linear or nonlinear spectral unmixing algorithms (Dickinson et al., 2001) can then be applied to the measured whole-blood Raman spectrum to identify the neutrophil component. A second approach according to the invention is to measure these spectra in an ex vivo sample of blood from the individual patient and then to use these as the reference or basis spectra to analyse subsequent in vivo spectra. A third approach is to identify one or more specific lines in the neutrophil Raman spectrum that are unique to these cells or at least have low intensity in other cells. The peaks seen in the neutrophil Raman spectra in FIG. 1 in the 3000-4000 cm⁻¹ region or around 2450 cm⁻¹ or around 1250 cm⁻¹ are examples of such spectral peaks. These features may then be isolated, for example, by also measuring the signal on either side of selected the peak(s), so that the non-neutrophil background can be subtracted from the total blood Raman signal. A fourth approach in the invention is to use various forms of chemometric analysis of the blood Raman spectrum to identify the neutrophil contribution.

One optical arrangement for a device according to the invention is as follows for the example of using a finger as the body part to be interrogated. The finger is first paced into an aperture within a light-tight enclosure that contains some or all of the optical components. Incident light at one or more wavelengths is directed to the finger by an optical system comprising lenses, mirrors or optical fibers. Some fraction of the Raman scattered light generated by the neutrophils in the blood within the finger at the time of measurement is then collected by suitable optics such as a combination of lenses or mirrors or optical fibers. It is passed through or reflected from one or more optical elements such as filters suitable to eliminate or substantially reduce the light that is elastically scattered from the finger. These elements also eliminate or reduce the background light signals coming from fluorescence generated in the tissue or fluorescence or Raman scattering that is generated in the optical components themselves, such as the cladding of optical fibers or the coatings on lenses or mirrors. Some filtering may be used prior to the light being incident on the finger.

The Raman light coming from the finger is spread across the full Raman spectrum. Methods to sample this spectrum include but are not limited to multi-band detection or full spectral scanning. The first approach using multiband detection is as follows. Before being detected on one or more photodetectors such as photomultiplier tubes or photodiodes, the light passes through or is reflected from one or more filters that have relatively narrow band in order to select one or more specific spectral lines that correspond to known features in the neutrophil spectrum. One or more other bands may also be selected that are not found or are at low intensity in the neutrophil spectrum. These signals are then used as the background signal generated in other tissue or blood components. In the second approach the full Raman spectrum of the light from the finger is measured. This can be done in various ways, for example using a scanning monochromator and a single photodetector or using a diffraction grating to disperse the light onto an array detector such as a photodiode array or CCD or CMOS array detector. This full-spectrum approach has the advantage of utilizing more information that can result in a more accurate separation of the neutrophil and non-neutrophil Raman signals, but is more complex.

Certain embodiments of the invention may use neutrophil fluorescence. In certain embodiments according to this aspect of the invention, the finger is located within a light-tight cavity as in the above Raman approach. A light source of one or more wavelengths to excite the neutrophil fluorescence is incident on the finger. Suitable wavelengths include but are not limited to wavelengths around the excitation maxima of the neutrophil fluorescence, such as around 290 nm, 350 nm, 410 nm, 450 nm or 500 nm.

The fluorescence light emitted by the neutrophils in the blood within the finger at the time of measurement may be collected by suitable optics such as a combination of lenses or mirrors or optical fibers. The fluorescence light from the finger may then be passed through or reflected from one or more optical elements such as filters or diffraction gratings suitable to eliminate or substantially reduce the excitation wavelengths that are elastically scattered from the finger and the background fluorescence coming from other components in the finger. The light is then detected by one or more photodetectors such as photomultiplier tubes, photodiodes or photodetector arrays such as photodiode arrays or CCDs or CMOS detectors.

Isolating the neutrophil autofluorescence from the fluorescence of other blood cells and plasma is similar to the above procedures for Raman measurements. In this case, however, since the fluorescence spectra are much less structured than the Raman spectra, it is generally advisable to identify spectral ranges, either of the excitation light or the emitted fluorescent light or both, where the neutrophil signal dominates over other blood components. If an exogenous fluorescent label is used that is preferentially associated with neutrophils over other blood components, then the spectral ranges corresponding to this label may be used.

It is recognized that a challenge in using in vivo embodiments of the invention is that the autofluorescence signal from the circulating neutrophils that can be detected outside the blood stream may be small due to the high absorption of light by haemoglobin, especially at shorter UV and visible wavelengths where the neutrophil autoflorescence is strongest. For this reason, an alternative embodiment of the invention for non-invasive measurements is to place optical fibers within a blood vessel, such as an assessable vein, as in the placement of a central line for patient monitoring. The fluorescence excitation light or the fluorescence emission may then be delivered to or collected from the blood, respectively. This in-line embodiment and the invention devices would allow the neutrophil fluorescence to be measured with reduced attenuation effects. The other aspects of the invention, including the light sources, filters, spectrometer, detectors and spectral analysis procedures, may be similar to those for the non-invasive technique described above. This approach could also be applied to the Raman method, but may be less desired, since the Raman spectra can be measured in the near-infrared spectral range where the absorption of light by haemoglobin and other tissue constituents is low.

### Isolating the Neutrophil Fluorescence or Raman Signals in Blood In Vivo.

The neutrophils are not the only source of fluorescence or Raman signals from the finger, since other blood or hard and soft tissues will contribute to any non-invasive measurement. The fraction of the optical signal represented by the neutrophils may be small, so that one aspect of the invention is to isolate the fluorescence or Raman signal that originates in the blood, including the signal from the circulating neutrophils, from that of the non-blood compartments of the finger. This can be achieved in one of several different ways, or by a combination of these.

One embodiment of the invention comprises a method to isolate the fluorescence or Raman signal from the blood is to utilize the pulsatile nature of temporal variations in the blood content in the finger due to the heart beat. In this method, a second optical measurement is made at the same time as the fluorescence or Raman signals are measured, most suitably over the same finger. This second measurement should be sensitive to the presence and concentration of the blood in the finger. A suitable approach is to measure the light that is diffusely reflected from or diffusedly transmitted through the finger at one or more wavelengths where these optical signals are substantially affected by the blood absorption. Suitable wavelengths include those where hemoglobin, in either the deoxygenated form (Hb) or the oxygenated form (HbO₂), absorbs light, such as in the wavelength range from about 600 to about 900 nm. Shorter wavelengths may also be used, depending on the depth or thickness of the tissue over which the measurements are made, with longer wavelengths being used where greater thickness or depth is required.

At any wavelength, it is well known that these diffuse light signals, R(λ) or T(λ), depend on both the absorption and elastic scattering coefficients at the wavelength λ and a number of methods are available to separate these two components, for example Doornbos et al. (1999). The diffuse light measurements can be made by illuminating the finger with either broad-band light or one or more selected wavelengths. The spectrum of light that is diffusely reflected from or diffusely transmitted through the finger is then altered due to the absorption by the hemoglobin. Hence, measuring R(λ) or T(λ) continuously or repetitively with time produces a time-varying signal that tracks with the pulsatile blood content of the tissue. As discussed above, this effect is utilized in the technique of pulse oximetry to measure the oxygenation of the blood for the purpose of monitoring the health of the patient. In certain embodiments of the present invention, the time varying diffuse reflectance or transmittance signal is used instead to isolate the fluorescence or Raman signals coming from the blood relative to the constant background signals coming from other tissue components. For example, during the systolic period of the heart beat, the blood content of the tissue would be high, so that the fluorescence or Raman signal from the blood, including from the neutrophils, would also be high. The reverse would the case during diastole, where the blood content of the tissue, and so the neutrophil fluorescence or Raman signals would be low. It is then clear that simultaneously monitoring the diffuse reflectance or transmittance signal from the finger enables the constant background due to fluorescence or Raman light not originating in the blood to be subtracted.

An additional method to isolate the fluorescence signal from the blood according to the invention is to artificially alter the blood content of the tissue, such as in the finger. The purpose is to induce larger time-varying changes in the blood content in the tissue those caused by the pulsatile circulation. In one method this is achieved by applying local mechanical pressure to the finger. Again, the fluorescence or Raman signal originating in the blood, including the neutrophils, will be high when the blood volume in the finger is high and vice versa. However, in this method a greater degree of modulation of the diffuse light signal can be obtained than in the method above using the natural pulsation. The pressure may be applied in a cyclical fashion, for example, as a continuous cycle of pressure waves such as sinusoidal waves or in a single or repeating ON-OFF manner. In any case, the fluorescence or Raman signals from the non-blood components in the finger will be essentially constant, while the blood fluorescence or Raman signals will vary with the applied pressure and so may be separated from the background.

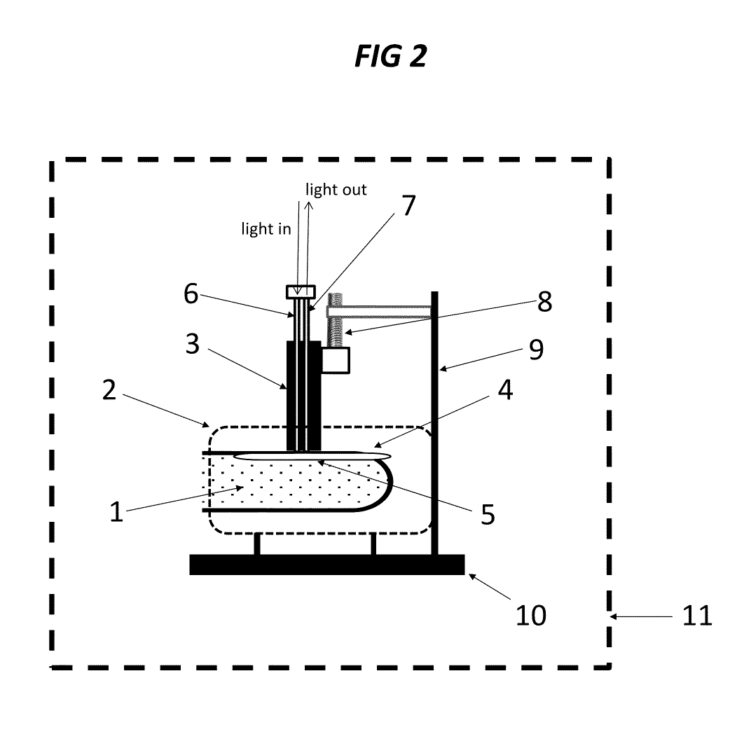

In one embodiment of a device according to the invention a method to make non-invasive in vivo measurements is shown in FIG. 2 for the purpose of illustrating the principle. The finger 1 is supported in a receptacle 2. A probe 3 is placed in contact with the finger nail 4. Optical measurements are made of the nail bed 5 using one or more optical fibers. In the example illustrated here one fiber 6 delivers light to the tissue while a second optical fiber 7 collects light from the tissue but this can be done in a single fiber or by multiple fibers. There are two aspects to the delivered and detected light. In the first aspect the delivered light is used to excite the fluorescence or to generate the Raman scattered light. In the second aspect the delivered light is used to generate a diffuse reflectance signal from elastic scattering. Local pressure is applied through the probe tip to the tissue using an actuator 8, shown here as a mechanical actuator. The actuator is fixed by a rigid holder 9 that in turn is fixed with respect to the base of the device 10 and a light-tight enclosure 11.

In this embodiment, the optical measurements are made by optical fibers incorporated into a probe that is in contact with the finger or, as shown the finger nail. The probe is then mechanically pushed against the nail so that part of the blood is forced out of tissue. By applying the pressure in a continuous cyclic manner or a single or repeated on-off manner and relating this to the corresponding fluorescence or Raman measurements taken at the same time (e.g. by analysing the time dependence of the signals or using an electronic technique in which the fluorescence or Raman signals are “locked in” to the reflectance signal as a reference), the fluorescence or Raman signal coming from the blood in the tissue can be isolated from the constant tissue background.

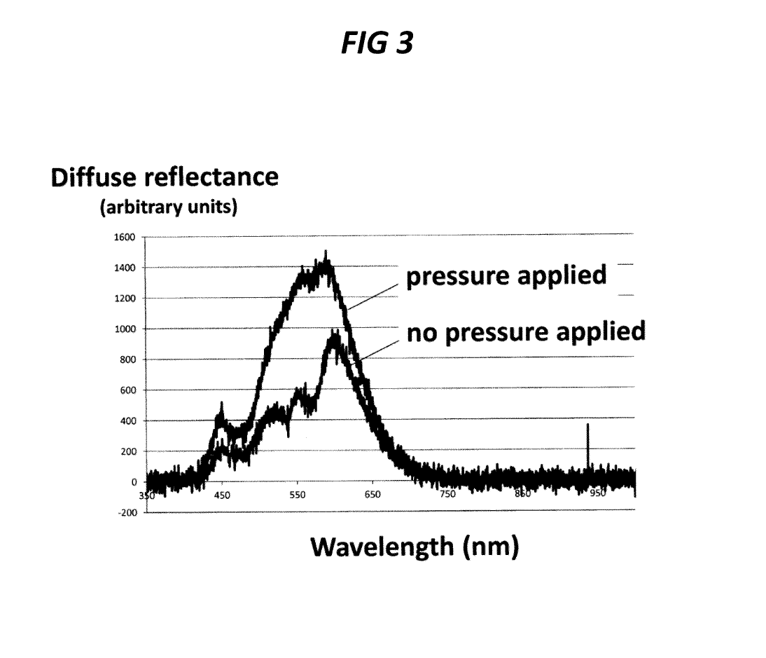

FIG. 3 shows one embodiment of the invention comprising a method for measuring the diffuse reflectance signal using a probe placed in contact with a finger nail. This figure demonstrates that the locally-applied pressure does push out a sizeable fraction of the blood and that this can be detected non-invasively as a change in the diffuse reflectance spectrum. In this figure the diffuse reflectance spectra were obtained using a contact fiberoptic probe placed in contact with a finger nail in an adult human subject, before and after application of applying slight pressure to the nail with the probe tip. In this case broad-band light was delivered through a 200 micron-diameter optical fiber and the diffuse reflectance was collected and sent to a spectrometer using a second 200 micron-diameter fiber with a center-to-center fiber spacing of approximately 500 microns. The well-known double peak in the HbO₂ spectrum at around 540 and 575 nm is seen here as dips in the reflectance spectrum. These dips are not so apparent in the spectrum when pressure is applied, showing this the change in the spectrum with pressure is due to reduction in the blood content of the tissue. Certain embodiments of the present invention allow objective and quantitative measurements of isolating the fluorescence or Raman signal of the blood in the finger from the signal of other tissue components. The spectra are shown in FIG. 3 in arbitrary units, but this can be converted into an absolute diffuse reflectance scale (0-100%) by an appropriate calibration procedure, for example, against a phantom of known diffuse reflectance at the wavelengths of interest.

This method of measuring the changes in diffuse reflectance or transmittance of the tissue due to local pressure may also be used to make other non-invasive measurements of blood in vivo. In this case the ‘in-blood’ part of the diffuse light signal is used, either by itself or synchronized with another measurement such as fluorescence or Raman. The clinical applications include measuring the hematocrit, measuring the concentration of other endogenous analytes or measuring the concentration of administered agents that are in the blood such as drugs.

Other embodiments of the invention comprising additional ways of artificially inducing a time-varying change in the blood content of the tissue, for example, by changing the temperature of the tissue, are envisioned. The principle of this method to isolate the fluorescence from the blood would be the same as described.

An additional method according to the invention comprises preferentially isolating the fluorescence signal that is coming from the blood relative to the signals coming from other components of the tissue in vivo is to make the measurements directly over a blood vessel lying near the surface of the tissue. Examples of suitable vessels include veins in the wrist on the back of the hand, the carotid artery in the neck or the retinal blood vessels. In the last example, the method could be incorporated in to existing ophthalmic instruments such as a fundus camera or digital retina scanner or optical coherence tomograph. Whatever the vessel used, the excitation light may then be directed only to the vessel and the fluorescence or Raman light would be collected only from the same area. This method can be combined with the above methods, so that the time-varying diffuse signal from the blood vessel would further increase the degree of isolation of the fluorescence or Raman signals originating in the blood.

Having thus isolated the fluorescence or Raman signal from the blood, further methods may be applied to remove or reduce the corresponding contributions coming from components in the blood other than neutrophils, such as the blood plasma or other blood cells. Some of these methods are presented above, including various methods of spectral analysis. One method is to use the specific fluorescence or Raman spectra of neutrophils. An alternative method is to use the fluorescence lifetime of the signals from neutrophils by using pulsed light and measuring the decay of the fluorescence signal as a function of time following the pulse. A further alternative is to illuminate the finger with excitation light that is intensity modulated at high frequency, typically in the range of tens or hundreds of MHz, and analysing the phase-shifted and intensity-demodulated diffuse signal, which also depend on the fluorescence lifetimes of the neutrophils. While these general methods are well known (Vetromile and Jameson, 2014), they have not been applied for the purpose of isolating the autofluorescence of neutrophils in the blood.

Another embodiment of the invention for using either the neutrophil autofluorescence or Raman signatures is to use a fluorescent or Raman marker that is specific to neutrophils relative to other components in blood. One example would be to administer by intravenous injection fluorescent or Raman-active agents or nanoparticles that bind preferentially to neutrophils compared to blood plasma or other blood cells. An additional embodiment uses fluorescent molecular beacons (Li et al., 2008) that are activated by neutrophils to a greater degree than by other blood components. These various markers can be targeted to the neutrophils using, for example, antibodies against antigens that are expressed on the surface of neutrophils.

These embodiments using the spectral or lifetime characteristics of the neutrophil fluorescence or Raman scattering or using markers to enhance the neutrophil signal relative to the signals from other blood components can be combined with the described methods to isolate the blood fluorescence or Raman signals from the other components of the finger using the natural or induced time-varying diffusely reflected or transmitted light from the finger.

### Quantifying the Neutrophil Fluorescence or Raman Signals in Blood In Vivo

The above methods allow the fluorescence or Raman signal from circulating neutrophils to be substantially isolated from other sources of background optical signals generated in the finger or other body part. Thereby, it would be possible to make a clinical interpretation as to whether the signal is relatively high or low compared to previous measurements in the same patient or compared to other normal individuals or individuals with sepsis, neutropenia or other medical conditions that cause substantial changes in neutrophil counts in the blood.

A further aspect of certain embodiments of the invention is to make the fluorescence or Raman measurement quantitative so that the concentration of the neutrophils in the blood can be estimated. This will provide information that is analogous to the values reported in the current clinical laboratory blood assays to assess conditions such as sepsis or neutropenia. As discussed above, for this purpose it is required to correct the measured fluorescence or Raman signal for the effects of light attenuation by the tissue.

One novel method of correcting for the light attenuation effects in vivo in the present invention is as follows. First, the diffuse reflectance or transmittance from the tissue is measured, where the signal is integrated over an area or volume of the tissue that is affected by the natural or induced changes in the blood content of the tissue, as described above, for example using local applied pressure. This will be illustrated for the case of fluorescence. The fluorescence is excited at wavelength λ1 and is detected at wavelength λ2. (In the analogous Raman implementation, these would be the incident and inelastically-scattered wavelengths, respectively.) In practice, more than one excitation or detection wavelength may be used, in which case the technique is extended to increase the accuracy of the neutrophil measurement. The corresponding fluorescence measurements made under the two conditions where the blood content of the tissue is minimum or maximum are then Fmin (λ1, λ2) and Fmax (λ1, λ2). The diffuse reflectance is also measured at these two wavelengths and conditions, giving Rmin (λ1), Rmin (λ2), Rmax (λ1) and Rmax (λ2), The corresponding diffuse transmittances may be used as alternatives to the diffuse reflectance. These various measurements are then combined to calculate the neutrophil content, knowing their fluorescence spectral characteristics.

The formula shown in Eq (1) is presented as an example of the analysis that may be performed to estimate the true blood fluorescence signal, Fb. This is a semi-empirical formula based on using the diffuse reflectance information. Other equations may be used, depending on the accuracy required for the measurement.

a) Fb=A·(Term1−Term2)/Term3 (1a)

where Term1=Fmin(λ1,λ2)/{Rmin(λ1)ⁿ¹·Rmin(λ2)ⁿ²} (1b)

i. Term2=Fmax(λ1,λ2)}/[Rmax(λ1)ⁿ¹·Rmax(λ2)ⁿ²]} (1c)

ii. Term3=1−RBF (1d)

Term 1 and Term 2 serve the function of correcting the measured fluorescence signals for the effects of light attenuation in the finger. Thus, if n1=n2=0.5, the correction corresponds to the geometric mean of the reflectance values at the two wavelengths. Different power-law indices, n1 and n2, may be applied to the reflectance signals measured at the fluorescence excitation and detection wavelengths.

In Term 3 the RBF represents the fraction of the blood content in the tissue under the applied pressure relative to the content without pressure. The values of the factors A and n1,n2 are determined in one or more of several ways or using a combination of more than one of these methods: firstly, by modelling the propagation of light in the finger at these wavelengths, for example using diffusion theory or Monte Carlo computer simulation; secondly by measurements on calibration phantoms that simulate the optical absorption and scattering of the tissue; thirdly, by calibrating the derived blood fluorescence against standard ex vivo measurements of the neutrophil concentration. The value of the factor A incorporates the light intensity incident on the finger, the efficiency of the light detection and other scaling factors where appropriate to the measurement. A preferred method for determining the values of factors A and n1,n2 comprises measurement on tissue-simulating phantoms, followed by validation in subjects where the neutrophil concentration is known by clinical laboratory tests in blood samples.

The RBF value may be estimated in at least two different ways. One method uses the changes in the diffuse reflectance or transmittance signals from the finger measured at the excitation and/or emission wavelengths and with and without the applied pressure, i.e. under the conditions of normal and reduced blood content in the tissue. The second method, which is a variant of the first, uses measurements of the diffuse reflectance or transmittance at the isobestic point of hemeglobin. The advantage of this is that it makes the estimate of RBF independent of the oxygenation status of the blood. Other wavelengths may also be used. The changes in the diffuse signals are related to the changes in optical absorption of the tissue due to altered blood content. The effect of this on the measured diffuse signals is forward modelled for a range of tissue transport scattering coefficients at the corresponding wavelengths, for example by diffusion theory or Monte Carlo simulation to generate a look up table or nomogram, from which the RBF is read off.

This value for RBF may then be inserted in equation (1a) to correct for the fact that not all of the blood is removed from the tissue by the local pressure.

The algorithm described in equation (1) is not the same as known ratiometric techniques in which ratios between fluorescence or reflectance measurements at different wavelengths or at different source-detector distances are used.

The formulas represented in equations (1a-1d) can be applied, for example, using different pairs of wavelength λ1 and λ2 in order to distinguish the neutrophil fluorescence from the contributions from other blood components. Similarly, techniques such as fluorescence lifetime can be used at these wavelength pairs to further enhance the separation of the neutrophil and non-neutrophil fluorescence signals.

The neutrophil concentration in the circulating blood may then calculated as

Cn=Fb/Fb-norm (2)

where Fb-norm is the known reference standard fluorescence for unit neutrophil concentration. It is clear that this refers to the neutrophil fluorescence measured ex vivo under known conditions and at the same fluorescence excitation and detection wavelengths as employed in vivo. Alternatively, the value of Fb measured in vivo at any one time is compared with the value at another time at which the circulating neutrophil concentration is known, either from current clinical-laboratory assays or by using the ex vivo method and device of the present invention.

To correct the measured Raman signals, R(λ) from the blood for the analogous effects of light absorption and elastic scattering by the finger, equation (1) may be appropriately modified, replacing the measured fluorescence signals by the measured Raman signals. In this case, if the Raman shifts are relatively small, it is possible to collapse the correction terms in Equations 1b and 1c to refer only to the measured reflectance or transmittance values at a single wavelength. Equations (1a-1d) are then applied as for the fluorescence method, and equation (2) is replaced by

Cn=Rb/Rb-norm (3)

where Rb-norm is the known reference standard Raman value for unit neutrophil concentration and Rb is the corrected Raman signal.

We next show an example of the optical layout of a device using the above concepts and methods to measure neutrophil fluorescence non-invasively in vivo.

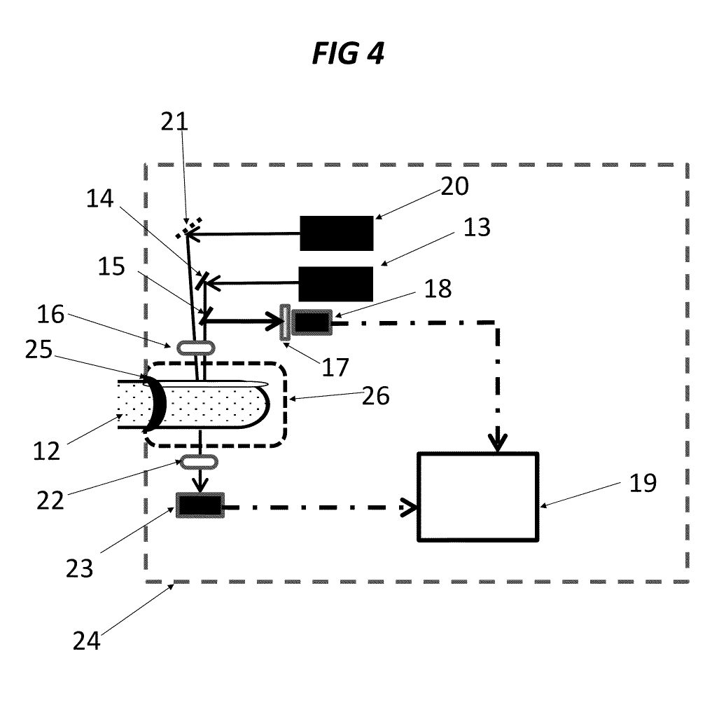

FIG. 4 shows a schematic of the optical elements for an embodied device configuration to measure the fluorescence and diffuse reflectance from a finger in vivo. Again, the nail bed of the finger, and the corresponding device are used here simply as an example of a tissue in which this can be done. It can be understood that similar principles would be employed for other body sites, with suitable changes to the layout of the optical elements to yield equivalent information. It can also be understood that the diffusely reflected light, as used for example to generate the data in FIG. 3, can be used instead of the diffusely transmitted light shown in FIG. 4 with suitable modifications to the optical layout and components of the device. It is further understood that similar principles can be used for making Raman measurements in vivo with suitable modifications to the optical layout and components of the device. For simplicity, this drawing also does not show the mechanical components for modulating the blood content by application of local pressure that are illustrated in FIG. 2.

In FIG. 4 the finger 12 is illuminated by light from a lamp, light-emitting diode or laser source 13 directed towards the finger by an angled mirror 14 after passing through a dichroic element 15 that transmits the fluorescence excitation wavelengths and through one or more lenses 16. A fraction of the fluorescent light generated in the finger, including by the neutrophils, is collected by the lenses 16, is reflected from the dichroic 15, and is transmitted through optical filters 17 to a photodetector 18 where it generates an electronic signal. The electronic signal is sent to a lock-in amplifier or signal sampler electronics 19. Alternatively (not shown), the fluorescent light from the finger is passed through a spectrometer or monochromator onto a photodetector to measure the fluorescence spectrum over a range of wavelengths. In a second function of the device, light at one or more red or near-infrared wavelengths from light source 20 is reflected from an angled mirror 21 through the lenses 16 to illuminate the finger over an area approximating that of the light from source 13. The diffuse light transmitted through the finger is collected by one or more lenses 22 to a photodetector 23 where it generates a second electronic signal that is also sent to the lock-in amplifier or signal sampler 19. This signal serves to synchronize the fluorescence signal generated by photodetector 18 so that the time-varying part of the fluorescence signal can be separated from the nearly constant background non-blood components of the finger. In an alternative configuration (not shown), the diffuse reflectance signal from the finger is measured instead of the diffuse transmittance. The parts 13-23 are placed within a light-tight enclosure 24 to eliminate ambient or stray light. The finger is placed through a flexible port 25 into a transparent receptacle 26 inset into the enclosure. The port limits the entry of ambient light from the surroundings into the instrument. The receptacle supports and immobilizes the finger during the measurements. In an alternative arrangement, the light may be delivered to or collected from the finger using optical fibers, to allow remote use of the device.

### Methods and Devices to Quantify Neutrophils Ex-Vivo

In certain embodiments of the invention, neutrophils can be quantified ex vivo by optical spectroscopy. Either fluorescence spectroscopy or Raman spectroscopy are used. As the haemoglobin in red blood cells is highly light absorbing, particularly in the UV and shorter visible wavelength ranges, in certain embodiments of the invention it may be necessary to remove this prior to spectroscopy measurements. In one configuration of the methods and devices, haemoglobin and/or cells are, therefore, removed from the blood sample prior to quantifying the neutrophils using spectroscopy.

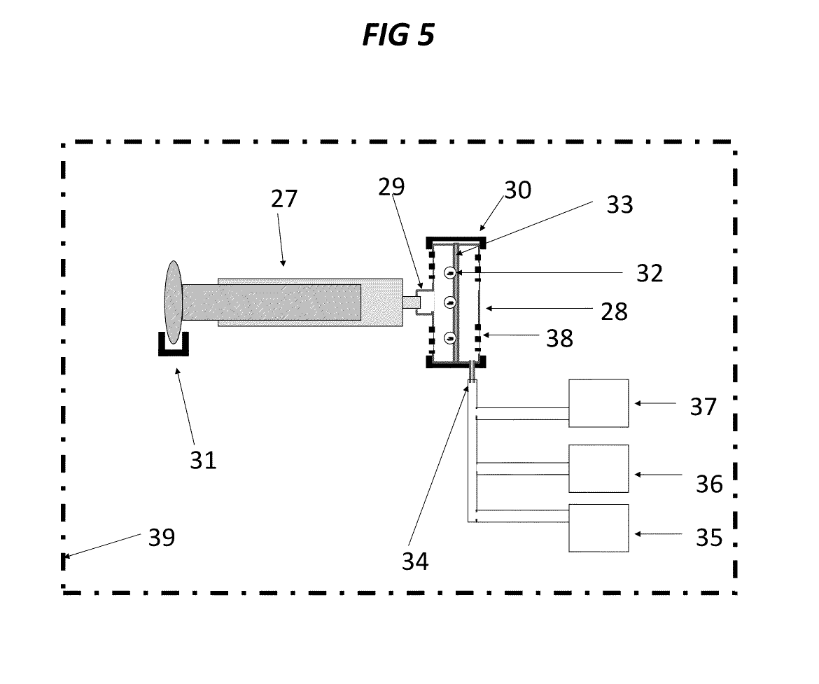

Certain embodiments of the present invention provide for ex vivo quantification of neutrophils in blood. A size filter may be used to provide a scaffold for partially-purified neutrophils, from which they can be quantified. The filter may be held within a cassette that allows efficient capture of the neutrophils on the filter and efficient removal of the fluid components of the lysed blood sample, while at the same time having one or more optical windows through which the spectroscopic measurements can be performed. This embodiment is illustrated in FIG. 5 which shows a schematic drawing of one example of an optical-filter cassette, shown in side view. The syringe 27 is attached to the optical filter cassette 28 via an entry port 29 with a locking mechanism. The syringe and filter cassette unit is placed in the device and the optical filter cassette is locked into place with cassette holders 30. The cassette holders keep the cassette and filter in set position of known geometry for optical analysis. A syringe driver 31 is clamped to the syringe plunger and moves to expel or take in solution into the syringe during the filtration and rbc lysis stages. Neutrophils 32 are trapped by the filter 33. Filter pores of about 10 microns allow for small molecules, such as haemoglobin and plasma components, to pass through the filter while trapping neutrophils on the filter surface for optical analysis. The filter opening on the side opposite the syringe attaches to a tube 34 in the device and is used not only to drain the filtered waste from the sample into a resevoir 35 but also to introduce ddH₂O from a resevoir 36 and PBS from a resevoir 37 into the sample for red blood cell lysis and washing. The cassette has one or more optically-transparent windows 38 that are indicated in FIG. 5 by dotted lines. These optical windows allow light to be directed into the cassette towards the filter 33 and fluorescent or Raman light from the cells 32 that are trapped on or in the filter 33 to exit from the cassette for spectroscopic detection and analysis. The cassette is placed within a light-tight box 39 that provides a controlled environment for the optical measurements. The cassette may also include a baffle (not shown) near the entry port 29 of the cassette that serves to spread the blood sample evenly across the surface of the filter, so that the optical probing can be done over any area of the filter, such as through one or more of the optical windows. The spectroscopy may be performed with the light sources and detectors on the same side of the size filter or on opposite sides. The materials and design of the cuvette are chosen to have minimal background contributions to the measured fluorescence or Raman spectra of the cell sample or to have distinct spectral contributions that can be separated from the measured spectrum.

It is clear that variations on this embodiment are possible. Certain aspects of this embodiment of the filter/optical cassette device embodiment are that 1). the neutrophils are trapped over an area of the size filter that is then optically interrogated through one or more optical windows in the intact cassette without removing the filter from the cassette, 2). the haemoglobin and plasma components of the blood are substantially removed from the blood sample, 3). the cassette does not substantially interfere with the spectroscopic measurement and 4). the cassette has a defined optical pathlength and allows a defined light source-detector geometry.

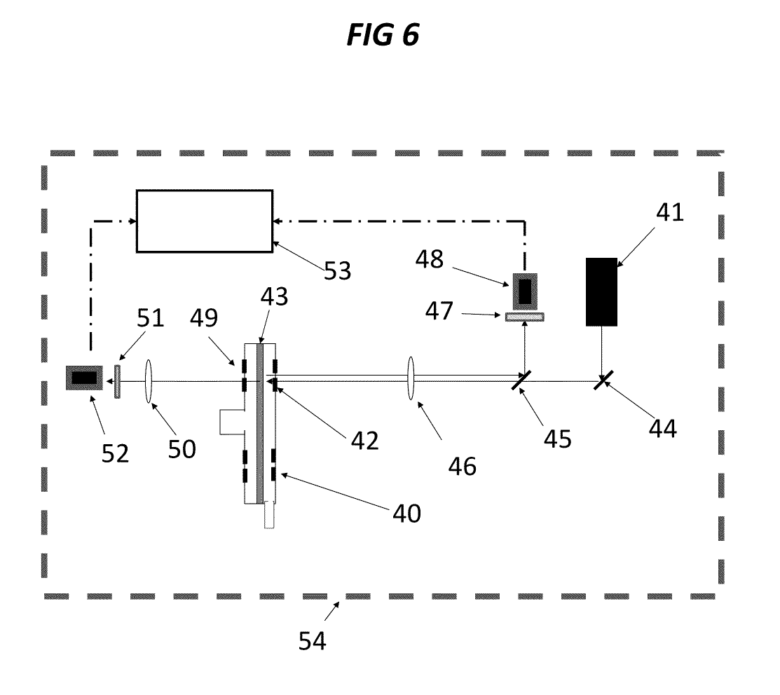

FIG. 6 illustrates one configuration of an embodiment of the optical set up for the spectroscopic measurements on the filter/optical cassette, for the case of fluorescence detection. With suitable modifications to the optical components, analogous arrangements may be used for Raman spectroscopy of the neutrophils trapped in the size filter. The cassette 40 is shown in simplified form here but is as described in FIG. 5. It is illuminated with a light source 41 directed through one or more optical windows 42 to the filter 43 by a mirror 44 after passing through a dichroic element 45 that transmits this wavelength through one or more lenses 46. Fluorescent light from the neutrophils exits the cassette via one or more optical windows 42 and is collected by the lenses 46, reflected by the dichroic 45 and transmitted through optical filters 47 to a photodetector 48 where it generates an electronic signal. This optical path corresponds to epifluorescence detection. An alternative detection path is also shown where the fluorescence light exits through one or more optical windows 49 on the opposite side of the size-filter and passes through lenses 50 and optical filters 51 to a photodetector 52. The optical signals are passed to an electronic device 53 suitable to process and store the signals for analysis. In either path, the single-element photodetectors 48 and 52 may be replaced by means to measure the fluorescence spectrum of the collected light such as a spectrometer. Parts 40-53 are placed within a light-tight enclosure 54 to eliminate ambient or stray light.

While the present embodiment of the invention focussed on ex vivo quantification of neutrophils, other size filters may be used that capture other target cells or molecules for subsequent spectroscopy analysis. Examples of clinical utility include lymphocytes in monitoring blood cancers or T cells for assessing infection.

A further aspect of the invention uses the filter/optical cassette to remove constituents in the blood other than haemoglobin.

A further embodiment of the invention uses the cassette without first lysing the red blood cells to remove the haemoglobin: this is applicable in the case where the optical spectroscopy measurements can be done even in the presence of haemoglobin, for example using wavelengths outside the haemoglobin optical absorption spectral range.

In one embodiment, the water or other lytic fluid, and subsequently the buffer, are added to the syringe or collection tube containing the blood sample and then this is attached to the filter/optical cassette. Alternatively, the cassette is attached to the syringe or collection tube and the water or lytic agent, followed by the buffer, is added through the cassette itself in the reverse direction. In another configuration the blood sample is injected into a second container and mixed with the water or lytic agent and subsequent buffer before being forced by pressure through the cassette. An additional wash can be used by forcing saline or physiological buffer through the cassette to ensure that all haemoglobin and cell debris are removed.

In another included configuration, optical labels are added to the sample either before or after red blood cell lysis.

An extension of the filter/optical cassette is an embodiment comprising a cassette having several size filters, each of which traps specific cells or components over a different limited segment of the filters. The spectroscopic “read out” is then accomplished by interrogating each segment, either in parallel using multiple light paths or sequentially. One example configuration is analogous to a “pie chart”. A second configuration divides the filter into quadrants.

In some embodiments of the invention, the neutrophils could be released from the filter, for example by reverse flow of a fluid through it, and subsequently measured spectroscopically. However, a clinical advantage of the present invention is to minimise or eliminate operator intervention as much as possible and this step is not necessary in all embodiments of the invention.

Another embodiment of the invention is to obtain clinically valuable information by performing optical spectroscopic measurements on the material that is not trapped by the size filters, such as the haemoglobin component, by incorporating a separate optical channel into which this material is passed.

Another embodiment of the invention uses filters to separate the various blood components that are based on physical or chemical characteristics of the components other than or in addition to size, including for example, surface electrical charge, chemotaxis, antibody binding, ligand binding and density separation.

In one configuration of the device two-photon optical spectroscopy may be used.

In another configuration of the device the collection tube and sample are connected to an automated device that attaches a new cassette for every sample.

In another embodiment, substances other than blood are collected and measured. This includes but is not limited to other biological material such as pleural aspirates, urine, stool, cerebral spinal fluid, ascites, joint aspirates, abscesses and fluid collections. The filter/optical cassette size, materials and configuration are then modified to be suitable for these samples.

### Variations of the Methods and Devices of the Invention

In certain embodiments the devices of the invention may be portable and able to be brought to the patient's bedside, to the patient in the community or any other location. It is envisaged this device would be used in a clinical environment to rapidly quantify neutrophils. It would be used as important tool to help identify patients with infections. It is envisaged this device will increase the rate of sepsis detection while reducing the need for invasive blood collection for neutrophil quantification.

Embodiments of the invention may also be used help identify patients with infection such as sepsis/SIRS at early stages and facilitate the initiation of early treatment. We envisage that this device will decrease morbidity and mortality.

Embodiments of the invention may also be used in multiple clinical applications and we provide here examples of some applications. Certain embodiments of this device will be used in the emergency department. In patients who present to the emergency department a rapid assessment is essential to make informed clinical decisions. This device will provide valuable information rapidly and alert clinical staff if neutrophils are either high or low so that appropriate management and treatment can be started promptly.

Embodiments of the invention may also be used in hospital wards to monitor patients along with the routine observations/vitals that are monitored currently. Routine observations alert clinical staff to changes in physiology and allow appropriate management. Early recognition of deterioration allows for early re-assessment of the patient and their management. Hospitalised patients are at risk of deteriorating with their presenting condition but are also at risk of developing a hospital acquired infection or other medical conditions. The device will provide more thorough monitoring of the patients physiological status.

Embodiments of the invention may also be used in community medicine by medical staff (GP, nursing staff, etc.) to help perform an assessment and aid in the clinical management of patients. It is possible that the in-vivo embodiment would play a greater role in community medicine but this is not to limit the possibility that the ex-vivo embodiment could also be used in the community. We envisage the device will help with decisions on severity of infection and the appropriate place to manage the patient, whether that be in the community or in hospital.

Embodiments of the invention may be used in the diagnosis and monitoring of neutropenia/neutropenic sepsis. A range of medications and medical conditions can suppress haematopoiesis and cause neutropenia (eg. chemotherapy, bone marrow suppression). A neutrophil count below 1.8×10⁹/L is considered neutropenic. It is important to monitor the neutrophil count in this group of patients, as they are immunocompromised and the risk of infection increases as the neutrophil count decreases. It is a potentially fatal condition. A neutrophil count below 0.5×10⁹/L is considered severely neutropenic and these patients are at high risk of developing an infection and should be barrier nursed in a positive pressure single room to limit exposure to pathogens. A patient with neutropenic sepsis has a confirmed infection with neutropenia and prompt administration of antibiotics is required for these patients as they are immunocompromised. The device has a role in monitoring neutrophil counts in patients either at risk of becoming neutropenic or are already neutropenic. The device also monitors neutrophil counts to in response to medical management (eg GM-CSF or withholding chemotherapy). The device can be used in Emergency Departments to diagnose neutropenia in the acutely unwell patient who presents and has not yet been diagnosed with neutropenia. Early identification of neutropenia allows prompt risk stratification and has impact on clinical management of the patient.

In vivo embodiments of the invention may also be used in the paediatric and neonatal population where venapuncture and other invasive blood letting techniques are distressing and painful for the child and their family. The devices could be used instead of some invasive blood letting techniques.

Embodiments of the invention may also be used in obstetrics to monitor the fetus and the mother during labour. Early identification of mother and fetus neutrophil counts could alter management of the labour.

Embodiments of the invention may also be used to provide spectral measurements on samples to generate a profile to diagnose disease, as certain diseases have a unique optical fingerprint.

Embodiments of the invention may also be used to monitor other circulating cells and/or molecules and/or drugs. Examples include, but are not limited to, gentamicin, vancomycin, digoxin, theophylline, haemoglobin, lymphocytes, cancer cells and blood glucose. Embodiments of the invention may also be used for other bodily fluids or samples.

Examples of embodiments of the invention are provided of both the ex vivo and in vivo approaches of the invention in detail. It is to be understood that the invention is not limited in its application to the details of construction and to the arrangements of the components set forth in the following description or illustrated in the drawings. The invention is capable of other embodiments and of being practiced and carried out in various ways as described herein. Also, it is to be understood that the phraseology and terminology employed herein are for the purpose of the description and should not be regarded as limiting.

### EXAMPLES

### Example 1

### In Vivo Neutrophil Concentration Determination

Step 1. In one configuration of the invention, the in vivo measurement is taken on the patient's finger through the nail. If required to avoid interfering with the optical measurements, certain nail polishes, false nails or other cosmetic decoration are first removed.

In one configuration the patient's finger is placed inside device in a stable and comfortable position. A flexible seal around the exterior acts to block out external light during optical measurement. In another configuration, a remote probe connected to the device is attached to the finger for the same purpose.

The optical probe is moved to be in gentle contact with the finger nail.

Cyclical or ON-OFF pressure is applied automatically, either through the optical probe itself or by a separate contact device. Raman or fluoresence spectral measurements are made, with and without the application of pressure so that the signals from the blood can be isolated from the signals from other finger tissues.

The neutrophil concentration in the blood is calculated automatically by the device and displayed to the operator, who may be any member of the clinical team (nurse, doctor, paramedic, auxiliary staff).

In some embodiments a blood sample may also be taken and used to calibrate the in vivo spectroscopy measurement so that the neutrophil concentration in the blood may be calculated and displayed.

In some embodiments, if the finger nail is not suitable for measurements, the finger is positioned so that the optical and pressure probes are applied to a different part of the finger.

### Example 2

### Ex Vivo Neutrophil Concentration Determination

Step 1. A known volume of blood is withdrawn from the patient.

Step 2. If necessary, water or other agent and subsequent buffer are added to selectively lyse the red blood cells and release the haemoglobin.

Step 3. Pressure is applied to force the sample through one or more size filters, selectively trapping neutrophils in the size filters. The size filters are held in a cassette specially designed to allow efficient filtration while having one or more optical windows to allow efficient spectroscopic measurements.

Step 4. Fluorescence or Raman spectroscopic measurements are made on the intact filters that trap the cells of interest such as the neutrophils.

Step 5. Spectral features of neutrophils are identified so that the optical signal may be separated from the signals from other biomaterials on the filters.

Step 6. A calibration factor is applied, based on the analogous measurements performed on a series of blood samples of known neutrophil concentration in order to calculate the neutrophil concentration in the patient's blood sample.

Step 7. The calculated neurophil concentration value is displayed to the operator.

### Example 3

### A Method and Device for Ex Vivo Fluorescence Quantification of Neutrophils

Step 1. Blood is drawn from the patient into a syringe. This syringe is preloaded with an anticoagulant to prevent clotting.

Steps 2 and 3. The haemoglobin and other interfering components in the blood sample are removed by adding water or another lytic agent to the blood sample. Since red blood cells are more osmotically fragile than other cells they will lyse first and release the haemoglobin into the plasma. Lysis of the other cells, including the neutrophils, is minimized by then adding concentrated osmotic buffer to form a physiologically normotonic solution.

Step 4. The sample is passed through a 10 micron filter to remove the haemoglobin, plasma and other cells or cell fragments, while capturing an enriched, concentrated population of neutrophils and some other nucleated cells in the filter. In addition to eliminating or substantially reducing the confounding effects of hemoglobin on the spectroscopy measurements, the size filtering enhances the spectral signals from the neutrophils, through their being concentrated in a smaller volume than in the original blood sample.

Step 5. Fluorescence spectroscopy measurements are made while the cells are still attached to the size filter without the operator requiring to remove the filter and place it in the spectroscopy device. This is achieved by pre-mounting the filters in a cassette that enables efficient filtration while having one or more optical windows to enable efficient optical spectroscopy in a single device. The materials of the filters and cassette are chosen to have minimal fluorescence signals or to have such signals that are spectrally well separated from those of the neutrophils so that they do not interfere with the neutrophil spectroscopic measurements. The filter/optical cassette device also has a well-defined optical path length to allow accurate quantitative spectroscopic measurements.

Step 6. Fluorescence measurements are made on the cassette either in reflectance or transmittance geometry, or both.

Step 7. The fluorescence signals from neutrophils in the filter-trapped sample are identified using discrete wavelength characteristics or by spectral decomposition, unmixing or chemometric-type approaches. Since these measurements are performed ex vivo with the hemoglobin absorption substantially eliminated and the neutrophils concentrated on the filter, shorter wavelengths can be used for fluorescence excitation, such as 255 nm, 290 nm, 350 nm, 410 nm, 450 nm and 500 nm, but the invention is not limited to these wavelengths.

Step 8. In order to finally achieve the clinically-useful value for the neutrophil concentration in the blood, it is not necessary to isolate the blood-related signals or correct for the light attenuation by the tissue as is required in the in vivo technique. Further, since the ‘geometry” of the ex vivo measurement is well defined by the size and shape of the cassette and by the optical configuration used, one simple method to obtain the neutrophil concentration is to carry out the same procedure (Steps 2-7 of this Example) on blood samples of known neutrophil concentration. These samples may be drawn from other patients having a range of normal and abnormal concentrations, in which case the current clinical-laboratory assay techniques provide the known neutrophil measurements. An alternative is to “spike” the blood sample with known numbers of neutrophils before performing Steps 2-7. In either case, the results serve as the calibration standard for the patient measurement.

Step 9. The neutrophil concentration value is displayed to the operator, who may be any member of the clinical or technical team.

### Example 4

### A Method and Device for Ex Vivo Raman Quantification of Neutrophils

The procedures are the same as in Example 3 except that iRaman spetroscopy is performed in Steps 5-8 instead of fluorescence spectroscopy.

In this example, two different embodiments of the procedure may be used.

In one embodiment, the Raman measurements are made using near-infrared, visible or ultraviolet light and all steps are performed analogously to the fluoresence method in Example 3.

In a second embodiment the Raman measurements are made using near-infrared light but without performing Steps 2-4 of Example 3. In this case the Raman measurements are made on the whole blood sample.

### Example 5

### Ex Vivo Raman Method and Device from Blood Sample

Step 1. Collect 2 mL of blood in a syringe pre-loaded with 3.8 mg of dried EDTA anti-coagulant (a concentration of 1.8 mg/mL of blood).

Step 2. Attach the filter cassette to the tip of the syringe and load the cassette/syringe combination into the light-tight analyser. The filter cassette houses a 10 micron size filter and does not optically interfere with the neutrophil analysis. The cassette contains optical windows, one on either side of the filter. The optical windows are placed to allow for Raman interrogation of the neutrophils. The cassette inlet is built to lock onto the tip of a syringe. Pressure on the syringe forces the solution through the filter while capturing the neutrophils on the filter surface. The opening on the other side of the cassette attaches to the device and is used as an outlet for the filtered waste but is also used to inlet to add ddH₂O and PBS (see step 3).

Step 3. The red blood cells are lysed by water lysis. 8 mL of ddH₂O is injected into the sample and left to incubate for 15 seconds. As erythrocytes are more osmotically fragile than neutrophils, they will lyse in this brief period and the neutrophils will remain intact. 2 mL of 7.2% PBS is added to the sample to produce a physiologically isotonic solution and halt lysis.

Step 4. Pressure is applied to force the sample through the 10 micron filter to capture the neutrophils and other cells while allowing the lysed rbc haemoglobin to pass through the filter.

Step 5. Repeat water lysis with another injection of 8 mLs of ddH₂O through the filter to re-suspend the sample for and initiate erythrocyte water lysis for another 15 seconds. 2 mL of 7.2% PBS is added to the solution to halt water lysis by forming an isotonic solution.

Step 6. Pressure is applied to filter the solution again through the 10 micron filter leaving a neutrophil enriched sample adhered to the filter and the majority of haemoglobin will be filtered from the sample.

Step 7. The filter cassette contains optical windows compatible with Raman spectroscopy that allows optical interrogation of the filter. The neutrophils on the filter are illuminated at near-infrared light such as 785 nm.

Step 8. Raman scattered light from the neutrophils is transmitted through the cassette optical windows and passes through optical filters.

Step 9. The emission spectrum is collected by a Raman spectrometer where it is converted into an electrical signal (for example, from Betatek, Toronto, Canada). Neutrophil concentration will correspond to the intensity of the signal generated.

Step 10. Neutrophil Raman emission intensity is used to calculate absolute neutrophil concentration in the initial sample by comparing it to a calibrated concentration curve.

### Example 6

### Ex Vivo Fluorescence Method and Device from Blood Sample

Step 1. Collect 2 mL of blood in a syringe pre-loaded with 3.8 mg of dried EDTA anti-coagulant (a concentration of 1.8 mg/mL of blood).

Step 2. Attach the filter cassette to the tip of the syringe and load the cassette/syringe combination into the light-tight analyser. The filter cassette houses a 10 micron filter and the filter and cassette material does not optically interfere with the neutrophils analysis. The cassette contains optical windows on either side of the filter. The optical window are placed to allow for fluorescent interrogation of the neutrophils. The cassette inlet is built to lock onto the tip of a syringe. Pressure on the syringe forces the solution through the filter while capturing the neutrophils on the filter surface. The opening on the other side of the cassette attaches to the device and is used as an outlet for the filtered waste but is also used to inlet to add ddH₂O and PBS (see Step 3).

Step 3. The red blood cells are lysed by water lysis. 8 mL of ddH₂O is injected into the sample and left to incubate for 15 seconds. As erythrocytes are more osmotically fragile than neutrophils, they will lyse in this brief period and the neutrophils will remain intact. 2 mL of 7.2% PBS is added to the sample to produce a physiologically isotonic solution and halt lysis.

Step 4. Apply pressure to force the sample through a 10 micron filter which captures the neutrophils and other cells while allowing the lysed rbc haemoglobin to pass through the filter.

Step 5. Repeat water lysis with another injection of 8 mL of ddH₂O through the filter to re-suspend the sample for and initiate erythrocyte water lysis for another 15 seconds. 2 mL of 7.2% PBS is added to the solution to halt water lysis by forming an isotonic solution.

Step 6. Apply pressure to filter the solution again through the 10 micron filter leaving a neutrophil enriched sample adhered to the filter and the majority of haemoglobin will be removed from the sample.

Step 7. The filter cassette contains optical windows compatible with fluorescent spectroscopy that allows optical interrogation of the filter. The neutrophils on the filter are illuminated with 255 nm, 290 nm, 350 nm, 410 nm, 450 nm or 500 nm excitation light.

Step 8. Fluorescent emission from the neutrophils passes through the cassette optical windows and optical filters.

Step 9. The fluorescent light is collected by a spectrometer where it is converted into an electrical signal. Neutrophil concentration will correspond to the intensity of the signal generated.

Step 10. The intensity of neutrophil fluorescent emission is used to calculate absolute neutrophil concentration in the initial sample by comparing it to a calibrated concentration curve.

### Example 7

### A Method and Device Quantifying of Neutrophils Ex-Vivo Using Cell Surface Labels

Similar to Example 5 & Example 6, neutrophils are quantified ex-vivo. However, in this Example the neutrophils are labelled with one or more markers with known fluorescence or Raman signatures. An optically-active agent, such as an antibody-fluorophore conjugate or Raman-labelled antibody, is added to the sample. The marker may be preloaded in the syringe to allow for a brief period of incubation for the marker to bind. Addition of the water and buffer to the sample followed by filtration would have a dual purpose in this example, 1.) to lyse the red blood cells and remove haemoglobin as before and 2.) to provide a wash to remove any unbound label. Labelled neutrophils are quantified using the techniques described in Example 5 or Example 6.

### Example 8

### A Method and Device Quantifying Neutrophils Ex Vivo Using Phagocytosed Labels

Another alternative approach is to label neutrophils internally by incubating the blood sample for a brief period of time with a neutrophil phagocytosed optically-active agent, such as a fluorophore or any molecule with a known Raman spectrum. The label to be phagocytosed may also be preloaded in the syringe and washed after a brief period of incubation similar to Example 7. Neutrophil quantification is carried out using the techniques described in Examples 5 or 6.

### Example 9

### Method and Device of Ex Vivo Quanitification of Neutrophil in Capillary Blood Using Raman Spectroscopy

Step 1. In this configuration of the device, a slot compatable with a disposable filter is located on the exterior surface of the device. The device may contain a retractable covering that can be shut after a sample is obtained to block out exterior light during optical analysis. Load a disposable supported filter into slot of the device.

Step 2. Use a disposable capillary blood lancet (for example, from Owen Mumford, USA) to obtain a sample of capillary blood. The filter is designed to hold a 0.20 mL volume of blood when saturated. The filter is supported on three sides by plastic support but the fouth side is free to receive drops of blood. A common place to obtain the sample is on the finger.

Step 3. Draw the sample of capillary blood onto the filter thereby saturating the filter paper with capillary blood.

Step 4. Close the retractable covering on the device to block out light and allow for optical interrogation of the capillary blood saturated filter.

Step 5. The capillary blood on the filter paper is optically interrogated by Raman spectroscopy to calculate neutrophil concentration. The blood sample is illuminated at for example 785 nm to generate Raman scattered light from the neutrophils.

Step 6. The Raman emission from the neutrophils pass through a pass band optical filter and is measured using a Raman spectrometer (for example, from Betatek, Toronto, Canada).

Step 7. Intensity of the Raman emission spectra is used to calculate neutrophil concentration by comparing the intensity to a known concentration curve.

Step 8. The device for the user provides a neutrophil concentration.

### Example 10

### Method and Device of Ex Vivo Quanitification of Neutrophil in Capillary Blood Using Fluorescent Spectroscopy

Step 1. In this configuration of the device, a slot compatable with a disposable filter is located on the exterior surface of the device. The device may contain a retractable covering that can be shut after a sample is obtained to block out exterior light during optical analysis. Load a disposable supported filter into slot of the device. Either the medical staff or the patient can load a disposable supported filter into the device.

Step 2. Use a disposable capillary blood lancet (for example, from Owen Mumford, USA) to obtain a sample of capillary blood. The filter is designed to hold a 0.20 mL volume of blood when saturated. The filter is supported on three sides by plastic support but the fouth side is free to receive drops of blood. A common place to obtain the sample is on the finger.

Step 3. Draw the sample of capillary blood onto the filter thereby saturating the filter paper with capillary blood.

Step 4. Close the lid on the device to block out light and allow for optical interrogation of the capillary blood saturated filter.

Step 5. The capillary blood on the filter paper is optically interrogated by Fluorescent spectroscopy to calculate neutrophil concentration. The blood sample is illuminated at 255 nm, 290 nm, 350 nm, 410 nm, 450 nm or 500 nm to excite the neutrophil fluorescence.

Step 6. The fluorescent emission light from the neutrophils passes through optical filters and is measured using a spectrometer.

Step 7. Intensity of the Fluorescent emission spectra is used to calculate neutrophil concentration by comparing the intensity to a known concentration curve.

Step 8. The device for the user provides a neutrophil concentration.

### Example 11

### Raman Spectroscopy of Blood Components

Centrifuge 5 ml of whole blood.

Place the red blood cells phosphate buffered serum (PBS) and measure the Raman spectrum using 785 nm light from a diode laser, with the signal collected over 100 s in reflection geometry over the wavenumber range 400-4000 cm⁻¹.

Measure the Raman spectrum of the plasma in the same way.

Wash the remaining cells, including the neutrophils, in lysis buffer to remove as much as possible any residual red blood cells.

Place the remaining cells in PBS and measure the Raman spectrum in the same way.

Identify differences between the neutrophils and other blood components in the respective Raman spectra.

Although in this Example the blood components were first separated by centrifugation prior to measuring their spectra as shown in FIG. 1, this was for the purpose of demonstrating that their Raman spectra are distinct. In one embodiment of the invention, physical separation of the blood components is not required. Further spectral differentiation between neutrophils and other white blood cells, is also possible (Ramoji et al. 2012) if this is required in order to make the clinical diagnosis of, for example, sepsis.

1-31. (canceled)

32. A spectroscopic device for quantifying neutrophils using flourescence or Raman scattering.

33. A device according to claim 32 wherein the fluoresence is autofluorescence.

34. The device according to claim 32 wherein the neutrophils are first removed from a mammalian body.

35. The device according to claim 34 further comprising a filter for capturing neutrophils.

36. The device according to claim 35 further comprising a means for removing hemoglobin or plasma.

37. The device according to claim 32 wherein the neutrophils are measured in a mammalian body.

38. The device according to claim 37 wherein the device is non-invasive.

39. The device according to claim 38 that comprises a Raman spectroscope or a fluorescence spectroscope.

40. The device according to claim 37 that further comprises a means to isolate the spectral signals from circulating blood in a body part.

41. The device according to claim 40 that further comprises a means for mechanical pressure.

42. The device according to claim 41 that comprises a Raman spectroscope or a fluorescence spectroscope.

43. The device according to claim 42 that further comprises a means to measure diffuse light and a means to correct for the effects of light absorption and scattering in the body part.

44. The device according to claim 32 configured to diagnose a condition selected from the group consisting of infection, neutropenia and sepsis.

45. A cassette device comprising a filter and an optical window, said cassette device being suitable for use in the spectroscopic device of claim 32.

46. The cassette device of claim 45 further comprising a spectroscope.

47. The cassette device according to claim 46 for quantifying neutrophils.

48. The cassette device according to claim 46 wherein the device further comprises a Raman spectroscope or fluorescent spectroscope.

49. A method for quantifying neutrophils in a mammal using a spectroscopic device according to claim 32.

50. The method according to claim 49 further comprising analysing the Raman spectra or fluorescent spectra of the neutrophils.

51. The method of claim 49 further comprising using mechanical pressure.

52. A method of diagnosing sepsis comprising using a spectroscopic device according to claim 32.

53. A method for measuring the diffuse reflectance or diffuse transmittance signal comprising:
a) using an optical probe placed in contact with a finger nail or other body part;
b) applying pressure to the finger nail or other body part to reduce blood constituents;
c) obtaining the diffuse reflectance or transmittance spectra;
d) releasing the pressure;
e) repeating step c);
f) analysing the diffuse reflectance or transmittance data obtained in steps b-e) to determine the concentration of blood constituents.
